(19) United States
(12) Patent Application Publication
(10) Pub. No.: US 2015/0218461 A1
(43) Pub. Date: Aug. 6, 2015

(54) Methods for Changing Stability of Water and Oil Emulsions

(71) Applicant: THE UNIVERSITY OF WYOMING RESEARCH CORPORATION D/B/A/ WESTERN RESEARCH INSTITUTE, Laramie, WY (US)

(72) Inventors: John F. Schabron, Laramie, WY (US); Jeramie J. Adams, Laramie, WY (US); Joseph F. Rovani, JR., Laramie, WY (US); Jean-Pascal Planche, Laramie, WY (US)

(73) Assignee: The University of Wyoming Research Corporation d/b/a Western Research Institute, Laramie, WY (US)

(21) Appl. No.: 14/427,974

(22) PCT Filed: Dec. 20, 2012
(86) PCT No.: PCT/US12/71087

§ 371 Date: Mar. 12, 2015

Related U.S. Application Data
(60) Provisional application No. 61/700,090, filed on Sep. 12, 2012.

(51) Int. Cl. C10G 29/20; C07C 7/20; C10G 25/12

(52) U.S. Cl. CPC C10G 29/20; C10G 25/12; C07C 7/20

(57) ABSTRACT

At least one embodiment of the inventive technology may involve the intentional changing of the stability of an emulsion from a first stability to a more desired, second stability upon the addition of a more aromatic asphaltene subfraction (perhaps even a most aromatic asphaltene subfraction), or a less aromatic asphaltene subfraction (perhaps even a least aromatic asphaltene subfraction) to a emulsion hydrocarbon of an oil emulsion, thereby increasing emulsion stability or decreasing emulsion stability, respectively. Precipitation and redissolution or sorbent-based techniques may be used to isolate a selected asphaltene subfraction before its addition to an emulsion hydrocarbon when that hydrocarbon is part of an emulsion or an ingredient of a yet-to-be-formed emulsion.

This application is an international application and claims priority to and benefit of U.S. Provisional Application Ser. No. 61/700,090, filed Sep. 12, 2012, said provisional application incorporated herein in its entirety.

### BACKGROUND

The most aromatic asphaltenic components from oil, which may be the most polar and pericondensed aromatic asphaltenic components, may play a major role in stabilizing emulsions (a term including foams). The emulsions can be water-in-oil or oil-in-water emulsions, or complex (mixed) emulsions. Asphalt foams are water vapor in oil foams which utilize non-foam emulsions with small amounts of water to make foams by heating the mixture to vaporize the dispersed water. The inventive technology described herein presents a new process for monitoring, predicting, formulating, controlling, stabilizing, and destabilizing water and oil emulsions or foams by evaluating or more generally, utilizing the subfractions of asphaltenes that affect emulsion stability. Particular embodiments may involve use of an on-column precipitation and re-dissolution analysis technique, and/or a sorbent-based technique for separating/isolating oil components. The inventive technology also describes new compositions and applications based on this new process, particularly in the field of asphalt workability.

Solubility separations can be used to isolate the most or more aromatic asphaltenic materials from oils, which can often then can be used to stabilize emulsions or foams in controlled formulations. Emulsions can be made more stable by adding such highly aromatic (which are often highly pericondensed and polar) components or surrogates thereof. Alternatively, emulsions can be destabilized by selectively removing such components from emulsions using sorbents. Such sorbents can include but at not limited to metals, ceramics, zeolites, clays, silica, limestone, glass, quartz, sand, alumina, or high surface energy carbonaceous materials such as petroleum coke, coal, charcoal, activated carbon, acids, bases, salts, or similar materials.

An adsorptive process can be used to isolate the most polar and aromatic components from oils. Portions of these materials can be selectively adsorbed onto high surface energy solid sorbents such as metals, ceramics, zeolites, clays, silica, limestone, glass, quartz, sand, alumina, or high surface energy carbonaceous materials such as petroleum coke, coal, charcoal, activated carbon, acids, bases, salts, or similar materials. They can then be desorbed by using a variety of solvents such as but not limited to aromatic hydrocarbons, acids or bases, carboxylic acids, pyrroles, aldehyde, ketones, alcohols, water, amines, pyridines, carbon disulfide, dimethyl sulfoxide or halogenated solvents. Emulsions and foams can be made more stable by adding such highly pericondensed and aromatic components or surrogates thereof. Less aromatic subfractions (which may be referred to as first asphaltene subfractions) can be isolated and used to destabilize emulsions, as desired. Generally, asphaltene subfractions of differing aromaticity may be used to change emulsion stability as desired; less aromatic asphaltene subfraction(s) (of sufficiently low aromaticity) may have an emulsion destabilizing effect while more aromatic asphaltenes of sufficiently high aromaticity may have an emulsion stabilizing effect.

Emulsions and foams can be used to significantly lower the temperature of application of asphalt-aggregate mixtures through warm mix or cold mix processes. Being able to produce asphalt emulsion or foams from an appropriately selected crude oil origin is an important stake for the highway industry which is trying to lower those application temperatures in order to reduce its energy consumption, its carbon footprint, fume emission and to improve its workers' safety and comfort. Embodiments of the inventive technology disclosed herein may offer a tool to predict the emulsion or foam ability of a given asphalt and relate it to its parent crude oil. It also provides a tool to formulate the emulsion or the foam itself.

Methods based on a new breakthrough on-column precipitation and re-dissolution separation technique developed at WRI offer significant advancement for the characterization of the pericondensed aromatic and polar materials and waxes in petroleum and residua. The methods provide solubility profiles for oil components. The development work and example separations with representative materials have been described in detail in U.S. Pat. No. 7,875,464, (an “Asphaltene Determinator™” patent), Schabron and Rovani 2008, Goual et al. 2008, and Schabron et al. 2010, each of which is incorporated herein in its entirety. The separations, in particular embodiments, are performed using an inert stationary phase consisting of ground polytetrafluoroethylene (PTFE). Although high-performance liquid chromatography (HPLC) instrumentation and detectors are used, there are no chromatographic interactions between the material being separated and the stationary phase. It is solubility based.

The WRI Asphaltene Determinator™ method which is based on the recently invented technique separates oils into four solubility fractions using step gradient solvent changes at 30° C.: heptane, cyclohexane, toluene, and methylene chloride:methanol (98:2 v:v) (Schabron et al. 2010). Other methods based on the technique such as re-dissolution with a continuous increase in solvent polarity/strength are possible also. The Asphaltene Determinator method allows for the measurement of the most polar and aromatic components in oil. In one example, these are the materials that elute with the last, or strongest solvent, methylene chloride:methanol (98:2 v:v). This method was used to evaluate emulsions involving petroleum materials. The results show that the most aromatic, including the most polar and pericondensed material in oil asphaltenes play a significant role in stabilizing water and oil emulsions, and that they are enriched in the emulsions (Schabron et al. 2012). Asphaltene Determinator technology may refer to technologies described herein, and/or disclosed and/or claimed in any one or more of the following: U.S. Pat. No. 7,875,464; U.S. Pat. No. 8,273,581; U.S. patent application Ser. No. 13/490,307; U.S. patent application Ser. No. 13/490,316; and U.S. patent application Ser. No. 13/600,039, each of which is incorporated herein in its entirety.

### Asphaltene Component Adsorption and Deposition

Asphaltenes are defined as a solubility class of associated chemical complexes which precipitate when petroleum is dissolved in a low polarity paraffinic solvent such as heptane, pentane, or isooctane, for example. A wide variety of polar and highly pericondensed aromatic molecules containing sulfur, nitrogen, and oxygen as well as metal complexes containing nickel, vanadium, and iron are concentrated in the asphaltenes. Asphaltenes can stabilize water and oil emulsions. These can be water-in-oil or oil-in-water emulsions, or mixed (complex) emulsions. Asphaltenes act as the major viscosity builders in oil. In catalytic upgrading processes such as hydrotreating, the presence of these materials can shorten catalyst life. The petroleum industry has developed various deasphaltening processes that involve dissolving oil in an excess of hydrocarbon solvent available in the refinery such as compressed propane, or a liquid aliphatic solvent stream, resulting in asphaltene precipitation. The disadvantage of such processes is the high cost of operation resulting from gas compression or solvent removal.

In prior work we have shown that asphaltene components of petroleum residua can adsorb onto on metal surfaces when the oil is heated to temperatures below the temperature at which pyrolysis cracking reactions begin (<340° C.). More deposits were observed on aluminum metal surfaces as the temperature of residua was increased from 100° C. to 300° C. (Schabron et al. 2001). The resulting asphaltenic material enriched in Ni and V was observed to deposit as dark spots on stainless steel and aluminum surfaces, but not on a non-polar polytetrafluoroethylene (PTFE) surface. This phenomenon appears to be due to the partitioning of the intermediate polarity material surrounding the aromatic asphaltene component molecules into the oil matrix solution, exposing the highly pericondensed aromatic or polar material. The pericondensed aromatic or polar material can flocculate and/or adhere to the polar metal surface. This is a cause of heat-induced fouling of pipes and heat exchangers in refineries.

The Asphaltene Determinator on-column precipitation and re-dissolution method, in embodiments, involves analytical scale precipitation of asphaltene components from oil within a column packed with ground inert PTFE using a heptane mobile phase (Schabron et al. 2010). The precipitated material may be re-dissolved in three steps using solvents of increasing solubility parameter: cyclohexane, toluene, and methylene chloride:methanol (98:2 v:v). The amount of asphaltenes (heptane insolubles) and the Total Pericondensed Aromatic (TPA) content can be determined in less than an hour. It was observed in the development work for the method that glass wool or glass beads strongly adsorbed asphaltene component molecules once they are separated from other peptizing molecules in the oil (Schabron and Rovani 2008). This observation of an undesired effect in the analytical method reinforced the concept of the possibility of asphaltene component molecule removal by adsorption onto a sorbent. In addition, the Asphaltene Determinator method also is ideally suited to evaluate the efficiency of removal of pericondensed aromatic molecules in sorbent-based asphaltene removal technology.

It is generally assumed that highly aromatic, polar and pericondensed material in oils are solubilized by intermediate polarity peptizing molecules present in the oils, but that when these structures are disrupted using heat, sorbents, or chemical treatments, the highly aromatic (perhaps also highly pericondensed molecules) become depeptized and they can then self-associate to form large insoluble pre-coke and coke complexes. The surface energy of the pericondensed material is the highest of any component in oil (Pauli et al. 2005). This and other observations related to heat-induced deposition have led us to discover that the most pericondensed, viscosity building aromatic structures could be selectively removed from oil by, in certain embodiments, heating or pre-treating the oil and exposing it to high surface energy polar or highly aromatic sorbent material. The resulting oil would be deficient in the most refractory polar and pericondensed aromatic structures and the product oil is more stable and less viscous than the original oil. The pericondensed material adsorbed to the sorbent can be desorbed by contact with a solvent(s) (e.g., via solvent rinsing), and these non-surfactant highly polar pericondensed aromatic materials can be used to stabilize water and oil emulsions or foams.

### BRIEF DESCRIPTION OF DRAWINGS

FIG. 1 shows conceptual water droplets in oil with emulsion stabilizing skin.

FIG. 2 shows a generic schematic for a refinery desalter unit and try layers, relative to which at least one embodiment of the inventive technology may find application.

FIG. 3 shows a toluene and water mixtures with fractions 1-4 material from preparative asphaltene determinator separation of lloydminster vacuum residuum asphaltenes added. from left to right, toluene and water blank (no emulsion); 5.1 mg of fraction 1 material (heptane soluble); 5.2 mg fraction 2 material (cyclohexane soluble), 5.1 mg fraction 3 material (toluene soluble) 5.1 mg fraction 4 material (methylene chloride soluble).

FIG. 4 shows a toluene and water blank mixture on the left and a water in oil emulsion between toluene and water stabilized by adding 5.2 mg of asphaltenic material desorbed from silica gel following sorbent treatment of Canadian Bitumen (Canmet Energy) on the right.

FIG. 5 shows toluene and water emulsions created by adding increasing amounts of Peak 3 (toluene soluble) asphaltene subfraction material from Lloydminster vacuum residuum n-heptane asphaltenes. From left to right: toluene/water blank mixture; 5.0 mg, 10.5, mg, 21.0 mg, 42.0 mg, 85.0 mg, 170 mg toluene soluble material added. Note that the oil in water emulsion on left becomes water in oil emulsion at about 43 mg material added.

FIG. 6 shows the effect of adding about 100 mg silica gel to a toluene and water emulsion stabilized by 13 mg of the most polar and pericondensed Peak 4 material from a preparative Asphaltene Determinator separation of Lloydminster vacuum residuum asphaltenes: left—no silica gel, right—100 mg silica gel added.

FIG. 7 shows the effect of adding silica gel to a toluene and water emulsion stabilized by 5.1 mg of the most polar and pericondensed aromatic Peak 4 material (methylene chloride soluble fraction) from a preparative Asphaltene Determinator separation of Lloydminster vacuum residuum asphaltenes: left—toluene and water; middle—no silica gel; right—silica gel added.

### BRIEF DESCRIPTION OF THE PREFERRED EMBODIMENTS

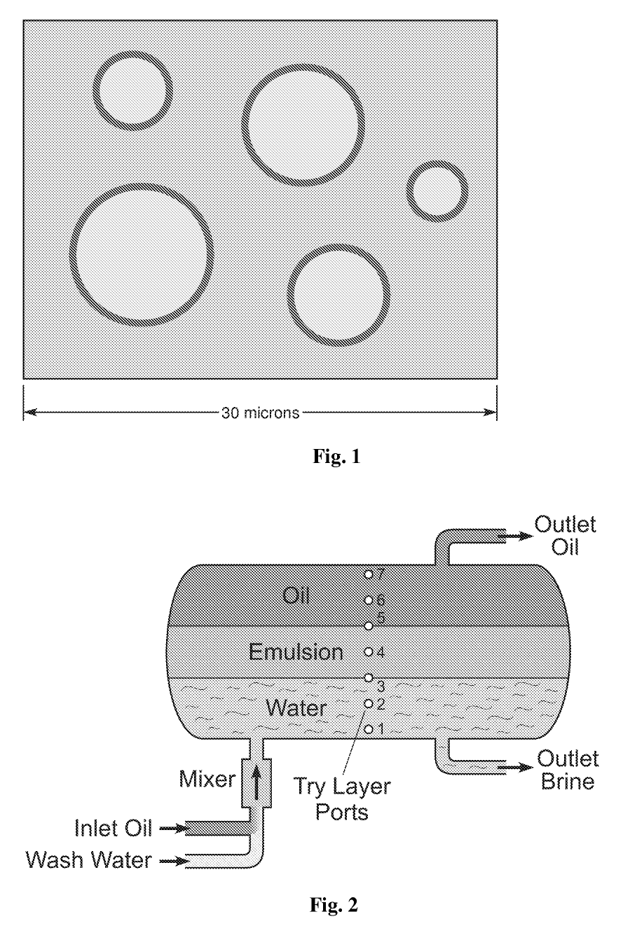

As additional background, components in the oil, especially the asphaltenes can affect to the emulsion stability. The emulsions can be water-in-oil, or oil-in-water, or mixed, depending on the process. Refinery desalter emulsions are believed to be water-in-oil emulsions that consist of spherical particles of water, each surrounded by a shell which stabilizes the emulsion structure (FIG. 1). Spherical droplet diameters of 1-28 microns have been observed (Ortiz and Yarranton 2010). Asphaltenes have been shown to stabilize the emulsions, while intermediate polarity resins act as destabilizers (Spieker and Kilpatrick 2004). The emulsions also can be stabilized by fine inorganic particles such as clays in the oils (Menon and Wasan 1988, Sztukowski and Yarranton 2005). For enhanced oil or bitumen recovery, ionic surfactants such as amines and sulfonic acid surfactants as well as non-ionic and surfactants have been used. These are often oil-in-water emulsions. The emulsions must be destabilized before processing. Chemical demulsifies are often used. The use of a magnetic field has been described for destabilizing emulsions (Peng et al. 2012).

Asphalt emulsion technology has been discussed in detail in Circular E-C102 from the Transportation Research Board (2006). Asphalt emulsions also require the use of surfactants or emulsifiers that can be classified into anionic, cationic and non ionic depending on the charge their polar portion head has in water and on pH. Those emulsifiers are typically fatty amines, quaternary ammonium salts, fatty acids or phenols (TRB 2006). In an emulsion break test, silica flour is blended into an emulsion to cause a break due to a surface area effect (TRB 2006). Foamed asphalts are usually made directly by injecting water into hot asphalt or occasionally by adding surfactants in asphalt prior to water.

Prior U.S. patent art related to stabilizing and destabilizing petroleum emulsions deals mainly with various types of surfactants including non-ionic, anionic, cationic, and amphoteric. Only a few examples are provided here. For stabilizing asphalt and water emulsions, various types of surfactants are used (U.S. Pat. No. 8,114,927, and 7,700,672). Demulsifier formulations are found in U.S. Pat. Nos. 5,445,765 and 5,164,116. U.S. Pat. No. 8,124,183 describes the use of calcium chloride, calcium nitrate, aluminum chloride, and ferric chloride to break emulsions.

### Asphaltenes and Emulsions

Asphaltenes, more so than any other component within crude oil, have been shown to contribute to the stability of water and crude oil emulsions (Sztukowski et al. 2003, Wu 2003, Hemmingsen et al. 2005, Jestin et al. 2007). Certain surface active asphaltene molecules or supramolecular asphaltene aggregates generate frameworks responsible for the stabilizing of these emulsions (Jestin et al. 2007, Czarnecki 2009, Czarnecki 2012). Upon agitation these networks concentrate at oil and water interfaces to produce either oil-in-water or water-in-oil emulsions. It is known that not all types of asphaltene molecules are responsible for stabilizing emulsion interfaces (Czarnecki and Moran 2005, Czarnecki 2012). The less aromatic resinous material in the oils or the asphaltenes can help solvate the aggregated material back into the oil phase and away from the interface, causing emulsion destabilization (Alvarez et al. 2009).

It is well known that small solids, especially those with high surface energy, concentrate at organic liquid and water interfaces to stabilize emulsions. Those emulsions are also called Pickering emulsions (Hannisdal et al. 2006, Wikipedia 2012). The size of the particles can have a direct effect on the stability of the emulsions: smaller particles create more stable emulsions.

It is also known that the adsorption of asphaltenes on minerals and reservoir rocks decreases when the surfaces are coated with water (water wet) but that asphaltene adsorption continues despite the buildup of several ordered layers of water at the surface (Collins and Melrose 1983). In a previous study designed to investigate enhanced oil recovery methods, a variety of oil/water emulsions were prepared using neat brine and dilutions of brine as the aqueous component of the emulsions to evaluate the effect of salinity on the quality of oil produced after emulsions were created and broken. The emulsion oils were characterized using the Asphaltene Determinator, and the results indicated distinct behavioral trends particularly in the asphaltenes component of the oil (Rovani et al. 2009). The results suggested for the first time that the Asphaltene Determinator could be applied in the design of core flood investigations to help understand the complex chemical interactions that occur in underground oil, water, and rock during secondary oil recovery waterflood operations.

### Asphaltene Removal

There is a great deal of prior art in U.S. patents that describe various approaches for removing asphaltenes from oil. Only a few examples are described here. U.S. Pat. No. 7,981,277 is a recent one that describes asphaltene removal using solvent precipitation. U.S. Pat. No. 4,888,108 describes the agglomeration of asphaltenes during solvent precipitation. Solvent deasphaltening in the presence of inorganic salt flocculating agents is described in U.S. Pat. No. 4,525,269. Asphaltene separation by cooling and crushing the solid frozen oil mixture, oil followed by particle size separation is described in U.S. Pat. No. 4,498,971. U.S. Pat. No. 4,765,885 describes a reaction of oil with sodium silicate for extracting asphaltenes into an aqueous phase, where the agglomerate and complex with metals present in the oil and settle to the bottom of a vessel. U.S. Pat. Nos. 4,514,287 and 4,424,114 describe the use of acidic compounds such as transition metal oxides to selectively remove the basic components of oil and asphaltenes by catalyst selective adsorption. U.S. Pat. No. 4,006,077 describes removal of metal containing compounds from asphaltene-containing oils using sorptive attapulgus clay. PCT App. No. US2012/021317 also describes asphaltene removal technology and, like the other patent documents mentioned herein, is incorporated herein in its entirety.

### Refinery Desalter Emulsion Study

The solubility subfractions of asphaltenes that contribute to water/oil emulsion stability were evaluated in a recent study, which is described below (Schabron et al. 2012). This is a major issue in oil production and enhanced oil recovery, where emulsion formation is desired in many cases, followed by deliberate destabilization of emulsions, and in refineries where the oil is washed with a water-based solution to remove salt and sediment, where persistent pudding-like emulsions are undesirable. In the study described below, the components of various oils that appear to stabilize oil and water emulsions were evaluated.

Before an incoming crude oil is distilled in a refinery, the first step is to remove salt and sediment using a fresh water-based wash. The separated salt and sediment emerge from the unit in an aqueous outlet brine bottom layer. A generic diagram of a desalter unit showing “try layer” sample port placement is shown in FIG. 2. For some oils and conditions, a large undesired middle emulsion phase forms which interferes with efficient operation of the unit. This results in a rag layer or pudding-like emulsion of oil and water which is persistent and difficult to break.

### EXPERIMENTAL

### Samples and Solvents

Crude oil samples and desalter emulsion samples were from petroleum refineries. Solvents and chemicals used in the study were reagent grade. Isolation of the most polar and aromatic (methylene chloride:methanol soluble) components from Lloydminster vacuum, residuum heptane asphaltenes is described in Schabron et al. 2010.

### Gravimetric Isolation of Asphaltenes

To prepare asphaltenes for the preparative Asphaltene Determinator separation, a sample of residuum or oil was weighed and mixed with an excess of heptane. The mixture was stirred overnight to allow full precipitation of asphaltenes. The mixture was then filtered using a medium frit (10 μm) sintered glass filter with repeated rinsing with heptane, and any residual solvent was removed using a vacuum oven at 110° C. The precipitate was dried and weighed.

### Saturates, Aromatics, and Resins (SARA) Separation

Open-column chromatographic separations of maltenes into saturates, aromatics, and resins fractions were conducted using a 400 mm×19 mm id glass column. The column was slurry packed with 35 g Aldrich grade 62, 60-200 mesh silica gel that had been activated overnight at 120° C. Sample amounts of 350 mg maltenes in heptane solution (1 wt. % loading) were place on the top of the column. Saturates, aromatics, and resins fractions were eluted with heptane, toluene, and toluene:methanol (80:20 v:v), respectively. The eluted fractions were rotovapped at 70° C. to near dryness, and then dried in a vacuum oven at 100° C. for 1 hour prior to weighing for gravimetric determination of the amounts in each fraction.

### Emulsion Centrifugation

Desalter emulsions were centrifuged in a manner similar to ASTM D-4007-02 without adding a demulsifier. The emulsions were centrifuged in an International Centrifuge; Universal Model UV, with 100 mL centrifuge tubes at 2,500 rpm with 16 inches tube tip to tube tip diameter while rotating. The centrifuging was performed in a series of three 10 minute intervals, with volume separations checked between each interval to ensure that there were no changes between the last two intervals.

### Analytical Asphaltene Determinator Separation

The on-column asphaltene precipitation and re-dissolution experiments were conducted using a Waters 717plus autosampler, a Waters 60F pump with a model 600 controller, a Waters 2489 ultraviolet/visible absorbance detector, and a Waters 2424 evaporative light scattering detector (ELSD) as described in Schabron et al. (2010b). Solutions of residua and asphaltenes in chlorobenzene were injected onto a 7 mm i.d.×250 mm stainless steel column packed with 0.25-0.42 mm ground polytetrafluoroethylene (PTFE) (40-60 mesh). The optical absorbance detector in dual wavelength mode at 500 nm and 700 nm was used to monitor the separation profile for a standard reference oil (Lloydminster vacuum residuum), which was injected daily to detect the possible onset of adsorption effects in the stationary phase. If adsorption is observed, the in-line pre-filter disc (Supelco 5-9271, 0.5 μm) and/or the column PTFE packing material is replaced to restore proper operation. Solvent flow rates were 2 mL/min with step changes between solvents. Peak area integrations were performed using Waters Empower software. ELSD and optical absorbance peak areas were electronically blank subtracted prior to integration to correct for small blank peaks due to the step gradient solvent changes. A 20 μL injection of 10% Lloydminster vacuum residuum is made daily as a QC check sample to ensure that there is no adsorption occurring on the column. Solutions of the sample oils and residua are prepared as 10% (w/v) or less solutions in chlorobenzene. Portions of 20 uL were injected in duplicate for the analytical scale Asphaltene Determinator separation. The optimized separation conditions are as follows:
Column: 7 mm id×250 mm stainless steel column
Packing: 40-60 mesh ground PTFE
Detectors:

Waters 2489 absorbance detector set at 500 nm and 700 nm

Waters 2424 evaporative light scattering detector (ELSD)
60° C. tube, 12° C. nebulizer, 35 psi nitrogen, gain=1
Solutions: Sample and QC solutions are 10% wt/vol in chlorobenzene
Injection amount: 20 μL
Solvents used for step gradient changes: n-heptane, cyclohexane, toluene, methylene chloride:methanol (98:2) (v:v), all at 2 mL/min
Step gradient times:

0 min. heptane

15 min. cyclohexane

25 min. toluene

35 min. methylene chloride:methanol (98:2)

45 min. heptane

60 min. next injection

All separation profiles are electronically blank subtracted prior to peak integration

Three representative sample sets were obtained from petroleum refinery operations.

These include incoming feed oils to the desalter, rag layer emulsions taken from the desalter units, and the desalted effluent oils, each set taken on the same day from a refinery desalter unit. One group of samples is for a light crude oil (API gravity ˜40), and another group is for a mixture of heavy and light crude oils (API gravity ˜25). A third group is for much heavier oil than the first two groups (API gravity ˜20). Differences in the Asphaltene Determinator solubility fraction profile distributions of the rag layer oil asphaltenes were observed in the current study when compared to the inlet and outlet oils. Results for the three sample sets are provided below.

### Light Oil Sample Set

Examples of results from the light oil group are provided in Tables 1-4. The incoming oil (Table 1) and desalted outlet oil (Table 2) appear very similar from the Asphaltene Determinator analyses corrected for ELSD volatiles losses. They also are similar in total heptane gravimetric asphaltenes content. However, the gravimetric asphaltenes from the desalted oil contain less toluene soluble and methylene chloride:methanol (98:2 v:v) soluble material than the asphaltenes from the feed oil (Tables 1-2). The gravimetric asphaltenes from the whole rag layer oil water emulsion (Table 3), which has the consistency of pudding, contain more methylene chloride:methanol (98:2 v:v) soluble asphaltene components than the asphaltenes from the incoming or desalted oils. To illustrate this, the area percent values for the 500 nm methylene chloride:methanol (98:2 v:v) peaks for the oils and 10-micron asphaltenes for the whole oil, the rag layer oil with water, and the desalted oil are summarized in Table 4.

The centrifuged emulsion contained 40% oil by volume, 54% water, and 6% sediment. The centrifuged rag layer supernatant oil (Table 5) contains about 20 times more gravimetric asphaltenes than the inlet feed oil or the desalted oil and these asphaltenes contain significantly higher methylene chloride:methanol (98:2 v:v) soluble asphaltene components than the gravimetric asphaltenes from the incoming oil. These results for an emulsion set with a single oil suggest that the most pericondensed and highest surface energy components of oil could be involved in stabilizing oil/water emulsions.

### Medium Oil Sample Set

Results for the two medium oil sets collected from a refinery desalter unit on the same day are provided in Tables 6-9. The rag layer emulsions were centrifuged in a manner similar to ASTM D-4007-02 as described above, without adding a demulsifier. The rag layer from Set 1 contains 60% oil by volume, 38% water, and 2% sediment. The rag layer emulsion for Set 2 contains 80% oil by volume, 14% water, and 6% sediment. The Asphaltene Determinator characterization data show that the inlet oils (Table 6) and outlet oils (Table 7) are similar in composition. The desalted effluent oil from Set 2 however has about half the amount of gravimetric asphaltenes as the incoming oil (Table 7). The explanation for this is not straightforward. It could be related to the timing of the sampling for the two materials. The gravimetric heptane asphaltenes from the whole rag layer oil/water emulsions, which have the consistency of pudding, contain significantly more methylene chloride:methanol (98:2 v:v) soluble asphaltene components than the incoming oils (Tables 6 and 8). This represents highly pericondensed and polar asphaltene material. The relative amounts of methylene chloride:methanol soluble material for the centrifuged emulsion oils are similar to the values for the inlet and outlet oils (Tables 6, 7 and 9). The supernatant oils from the centrifuged rag layer emulsions contain about half the amount of gravimetric asphaltenes relative to the incoming or effluent oils from Set 1 and about half the incoming oil from Set 2 (Tables 6, 7 and 9).

The area percent values for the 500 nm methylene chloride:methanol (98:2 v:v) peaks for the oils and 10-micron asphaltenes for the whole oil, the rag layer oil with water, and the desalted oil are summarized in Table 10. Enrichment of the most pericondensed and polar material represented by the methylene chloride:methanol soluble material in the rag layer emulsions suggest that the most pericondensed and highest surface energy components of oil could be involved in stabilizing oil/water emulsions.

### Heavy Oil Sample Set

For the heavy oil series, we were provided with samples from various try layer ports in a refinery desalter unit, as well as the incoming and desalted oils. The heavy oil sample set was for heavy oil (˜20 API gravity) samples collected from the “try layer” ports of a refinery desalter.

As with the light and medium oil sample sets, analyses were conducted using the Asphaltene Determinator separation and by gravimetric asphaltene precipitation followed by the Asphaltene Determinator. The samples that were analyzed included the whole samples shaken as received, the middle emulsion layers drawn from the samples, the oil from the centrifuged emulsions, and oily residue from the water which was evaporated from the centrifuged emulsions. The many tables of analysis results for this simple set are provided in Appendix A. The more significant results are provided in summary tables as described below. The oil, water, and sediment amounts in the emulsions obtained by centrifugation are provided in Table 11.

Data for the area percents of the methylene chloride:methanol (98:2) soluble material peaks detected by 500 nm absorbance are provided in Table 12. The amounts of this most polar and aromatic material are significantly higher in the whole samples containing emulsions and in the emulsions themselves. The try layer 5 and 7 oil samples, which did not contain emulsions, are more similar to the inlet and desalted oils. Relative volatiles-corrected ELSD area percents for the most pericondensed material are provided in Table 13. For the emulsions, the relative ELSD peak areas were corrected for both the water and volatile oils materials. The residues remaining from evaporation of the water layer after centrifuging contain a larger area percent of heptane insoluble material (asphaltenes) than the whole samples or emulsions.

Selected non-volatile component ELSD area percents are provided in Table 14. The material represented by non-volatile ELSD components is in the boiling range slightly above the nominal initial boiling point for atmospheric residua (>640° F., >340° C.). The ELSD area percents of heptane insolubles and the total pericondensed aromatic contents are highest for the evaporated emulsion centrifuged water residue material when compared with the data for the whole oils or emulsions.

The relative 500 nm absorbance detector area percents for the gravimetric asphaltenes from the oils and emulsions are provided in Table 15. The percents for the toluene soluble asphaltene components are for the most part similar for all the oils and emulsions. However, the relative peak areas for the methylene chloride:methanol soluble materials (Peak 4 materials) are significantly higher for the samples that contain emulsions.

These results show that the most polar pericondensed aromatic pre-coke material in the oils, represented by the methylene chloride:methanol soluble peaks, are enriched in the rag layer emulsion samples relative to the incoming oil or outlet desalted oils. The incoming crude and desalted crude oils are similar in composition to each other as expected. The oils from the emulsions after being centrifuged are somewhat similar to the incoming and desalted oils. The results support the hypothesis that the most pericondensed material in the oils contribute to rag layer emulsion stability.

As mentioned earlier, the present invention includes a variety of aspects, which may be combined in different ways. The following descriptions are provided to list elements and describe some of the embodiments of the present invention. These elements are listed with initial embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

### Applications

Typically various surfactant formulations are used to stabilize or destabilize emulsions. Asphaltene components are not typical surfactants which consist of hydrophobic and hydrophilic components in the same molecule (Czarnecki et al. 2012). However, some oils may contain carboxylic acids that can act as surfactants. It is known that certain components of asphaltenes can stabilize emulsions, and less polar resins components of oils can destabilize emulsions (Stanford et el. 2007). The work described above and in Schabron et al. (2012) confirms that a relatively small subfraction of asphaltenes, e.g., the more (at times the most) aromatic asphaltene component molecules (which may be very polar and pericondensed), are enriched in water and oil emulsions, and therefore can act a powerful agents for stabilizing emulsions. Conversely, by selectively removing the most polar and aromatic components from an emulsion using sorbents, the emulsion can be destabilized and broken. By developing approaches to apply the new asphaltene solubility profile separation methods to evaluate oil components that contribute to emulsion stability, emulsions can be better made, formulated, or destabilized.

In addition, asphalt paving processes involving warm mix, hot mix, cold mix, and foam emulsion formulations all utilize emulsion chemistry. Understanding the interplay of the asphaltene subfractions on emulsions will help to control foam or emulsion formation and stability, which is a key issue for asphalt emulsion chemistry. The invention is not limited to petroleum oils. Oils can include but are not limited to asphalts, distillation residua, processed oils such as from catalytic hydrotreating, tar sands oils, shale oils, coal oils, synthetic oils, biologically derived oils, modified and unmodified asphalt binders and formulations, roofing shingles, fuel emulsions, caulks, and sealants.

The invention is also not limited to downstream oil refining processes or asphalt emulsion formulations. Because the Asphaltene Determinator technique may also be used to evaluate the quality of oil produced by enhanced oil recovery techniques, the invention may lead to the development or refinement of chemicals such as emulsifiers, surfactants, or additives that may be used to “tune” the quality of the oil produced by water floods. With a better understanding of the complex chemistry that occurs in underground oil, water, and rock formations, it may be possible to use the invention to develop a technique to retain the most polar pericondensed components of the oil underground while producing higher-quality oil that is deficient in these materials.

On the other hand, aspects of the invention also involves adding asphaltenes or subfractions of asphaltenes to product formulations such as, but not limited to, petroleum or asphalts in order to make foams or any type of oil/water emulsions (O/W or W/O or W/O/W).

### Obtaining Asphaltene Subfractions

There are several ways that the more aromatic asphaltene subfraction (which is sufficiently high in aromaticity to achieve a desired increase in emulsion stability), described elsewhere herein as the second asphaltene subfraction, and which may be the more or most polar and pericondensed subfraction of asphaltenes, can be isolated. The on-column asphaltene precipitation and re-dissolution technology can be used to obtain asphaltenes or asphaltene solubility subfractions for use in stabilizing water-in-oil or oil-in-water emulsions or complex emulsions. They can also be separated further by the solubility separation of asphaltenes using the in-vessel material generation technology described in U.S. Pat. No. 7,875,464 and continuations thereof. In certain embodiments, asphaltene are precipitated in an inert stationary phase using a low polarity alkane solvent, for example, and then re-dissolved all at once or in portions with a solvent or solvents of higher polarity. Manual asphaltene precipitation and partial re-dissolution using various solvent mixtures can also be used to isolate asphaltene subfractions for use in stabilizing emulsions. The sufficiently aromatic, perhaps the most polar and pericondensed, portion of asphaltenes can also be isolated by selective adsorption from oil or emulsions onto sorbents which can be desorbed using various strong chromatographic solvents. The isolated asphaltenic material can be used to stabilize water and oil emulsions.

### Emulsions

Addition of asphaltenes or asphaltene subfractions such as the most polar and aromatic components of asphaltenes or surrogates thereof can be used to stabilize water and oil emulsions or foams/froths (the term foam will further be used only). One application of the invention includes adding asphaltenic oil components to emulsion formulations which result in stable emulsion or foam formation. Another application involves treating or removing the asphaltene components by selective adsorption to destabilize or break emulsions in oil production processes. The technology can also be used to predict, monitor, and destabilize undesirable emulsion formation in pipelines or in refinery desalter units. The technology can also be used to formulate emulsions or foams for warm mix or cold mix asphalt paving or overlay operations.

The on-column asphaltene precipitation and re-dissolution technique can be used to evaluate and predict the propensity of an oil to form or resist formation of water-in-oil or oil-in-water emulsions with aqueous phases such as water, salt water, and brine systems. The technique can also be used to monitor the use of additives, asphaltenes, and asphaltene subfractions to create or stabilize, or alternatively to destroy or destabilize emulsions.

The current configuration for the preparative Asphaltene Determinator developed at WRI which uses an inert stationary phase for a solubility separation, allows for the separation and collection of four distinct asphaltene fractions of increasing polarity and aromaticity in certain embodiments. In one study, the fractions were eluted with heptane, cyclohexane, toluene, and methylene chloride, respectively (Schabron et al, 2010). These four solubility defined subfractions of asphaltenes have distinct physicochemical differences. With increasing solubility parameters of the dissolution eluting solvents there is an increase in aromaticity and polarity. Other solvents or combination of solvents can be used to separate similar fractions using the technique

More resinous (less polar and less aromatic) asphaltenes subfractions which may be from the heptanes or cyclohexane fractions and which may be described as a first asphaltene subfraction can be added to emulsions to terminate supramolecular aggregation giving smaller discrete aggregates. These less aromatic resins materials can disrupt the ability of the supramolecular framework to dynamically make and break bonds between other aggregates that stabilize the networks at the oil and water interface. This approach could be useful for treating and destabilizing emulsions in oil production or refinery desalter units. They also could be used at other key points along the production chain which currently use surfactant additives to mitigate unwanted emulsions in oil production operations.

Due to favorable aggregation, adsorption energy, surface energy, and bond forming sites the more polar and pericondensed aromatic asphaltene subfractions—that elute with toluene and methylene chloride, for example—can be used to enhance emulsions. These fractions can be used independently or combined with other additives to be blended in with other asphalt/asphaltene materials to increase emulsion stability.

Another part of the invention is that selective use of asphaltenes or asphaltene subfractions can be used to control the type of emulsion formed (oil-in-water vs. water-in-oil). This can involve adding different amounts of asphaltene materials, or different polarity and aromatic types of asphaltene subfractions materials.

Road asphalt and sealants, roofing asphalt and sealants, aerosol and non-aerosol sealants, and fuel oil emulsions can be formulated using the most aromatic subfractions (and possibly also the most polar subfractions) of asphaltenes, such as the toluene and/or methylene chloride soluble asphaltene subfractions after removal of the heptane and cyclohexane soluble subfractions, for example. Other similar solvent schemes can be used to separate asphaltenes into less aromatic pericondensed and more aromatic pericondensed aromatic asphaltene molecular constituents.

### Sorbents to Destabilize Emulsions

Sorbents can be used to destabilize the water and oil emulsions. Oil and water emulsions can be effectively destabilized by adding particles, preferably with high surface energy or charge, to adsorb asphaltenes from the water-water droplet interface onto the water-solid surface interface. If the particles are sufficiently large, they can adsorb asphaltenes, perhaps adsorbing a more aromatic subfraction, from the emulsion interface, resulting in breaking the emulsion and giving an asphaltene byproduct that can be separated out of the mixture by settling, flotation, or filtered, for example. The adsorbed asphaltenes from the interface can be rinsed off with organic solvents like toluene or methylene chloride regenerating the sorbent for further use. The rinsed asphaltenes can be used as feedstocks for road or roofing asphalt, sealants, fuel oil, or further upgraded by processes such as hydrocracking. They also can be used to stabilize emulsion for other applications. The sorbent can be one with high surface energy that is selective to adsorption of asphaltene component molecules such as highly aromatic (possibly also highly polar and pericondensed) molecules. Examples of the sorbents which may be particularly useful include but not limited to metals, ceramics, zeolites, clays, silica, limestone, glass, quartz, sand, alumina, metal oxides, alumina silicates, metal oxides impregnated on alumina or silica or zeolites or alumina silicates, or high surface energy carbonaceous materials such as petroleum coke, coal, charcoal, activated carbon, or similar materials. Other sorbents such as salts or acids or bases might be useful also.

### Experimental Results

To evaluate the emulsion stabilizing ability of the most polar and pericondensed material in oil, several experiments were conducted using toluene and water in 9-mL vials. Toluene and water are not miscible and they do not form a natural emulsion when shaken or blended together.

### Emulsions Stabilized by Preparative Asphaltene Determinator Subfractions

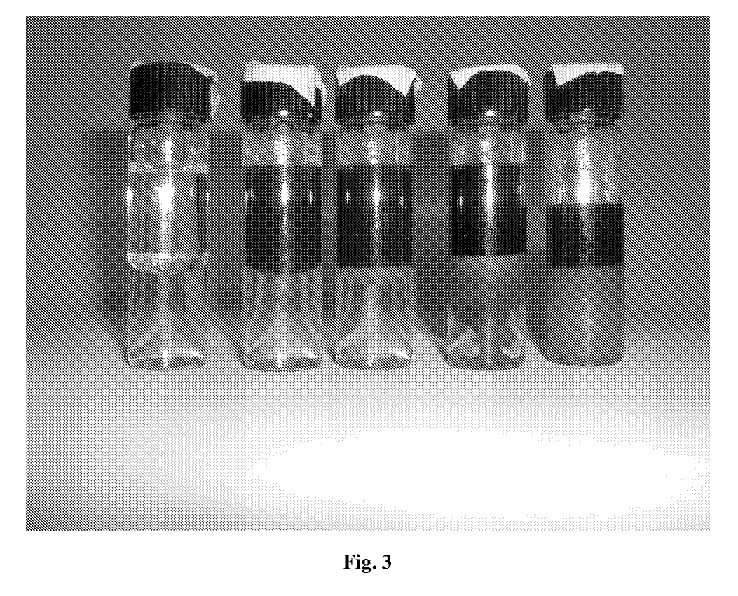

A preparative Asphaltene Determinator separation was conducted on 3.0004 g of n-heptane asphaltenes from Lloydminster vacuum residuum as described in (Schabron et al. 2010). Four solubility subfractions were obtained: Fraction 1 (heptane soluble, 0.1349 g), Fraction 2 (cyclohexane soluble, 0.7363 g), Fraction 3 (toluene soluble, 2.0875 g), and Fraction 4 (methylene chloride, 0.0401 g) soluble subfractions were obtained. These represent increasing polarity and aromaticity subfractions of asphaltenes. Portions of these asphaltene subfractions were added to mixtures of 3.5 mL toluene and 3.5 mL distilled water in 9-mL vials, and then these were agitated using a vortex mixture. The amounts of each of the solubility subfractions added were added were: Fraction 1, 5.1 mg; Fraction 2, 5.2 mg; Fraction 3, 5.1 mg; and Fraction 4, 5.1 mg. The results are shown in FIG. 3. No emulsion formed with the heptane soluble Fraction 1 material. A small amount of emulsion formed with the cyclohexane soluble Fraction 2 material. A larger amount of emulsion was evident with the toluene soluble Fraction 3 material. The greatest amount of emulsion was formed with the methylene chloride soluble Fraction 4 material, which consists of the most polar and pericondensed aromatic material component of the asphaltenes. As evident in FIG. 3, a small portion of the Fraction 4 mixture was lost during handling.

### Emulsion Stabilized by Desorbed Asphaltene Subfraction

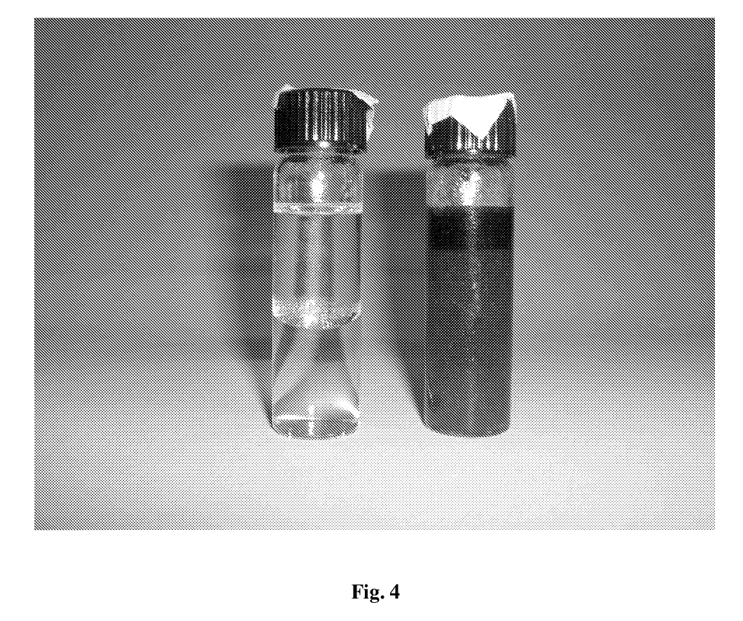

A 25.0 g portion of Canadian Bitumen (Canmet Energy) was mixed with 9.4599 g activated silica Grade 646 (35-60 mesh) at 300° C. in a sealed vessel for 4 hours with agitation. This treatment results in heat-induced adsorption of very polar and pericondensed asphaltene material onto polar surfaces, such as aluminum, steel or silica gel (Schabron et al. 2001). The method that was used to isolate the asphaltene material resulted in the emulsion used in FIG. 4 may be as described in PCT App. No. US2012/021317, said application incorporated herein in its entirety. The asphaltenic material was desorbed from the silica gel. A portion of 5.2 mg of this material was added to a 9-mL vial and suspended in 3.5 mL toluene. The suspension was mechanically shaken for 30 minutes until it dissolved, 3.5 mL distilled water was added, and the mixture was agitated using a vortex mixture for 60 seconds. A significant emulsion resulted (FIG. 4). The results show that heat-induced deposition of asphaltene components on to sorbents can be used to isolate material from oil that can be used to stabilize water and oil emulsions.

### Oil-in-Water and Water-in-Oil Emulsion Formation Control

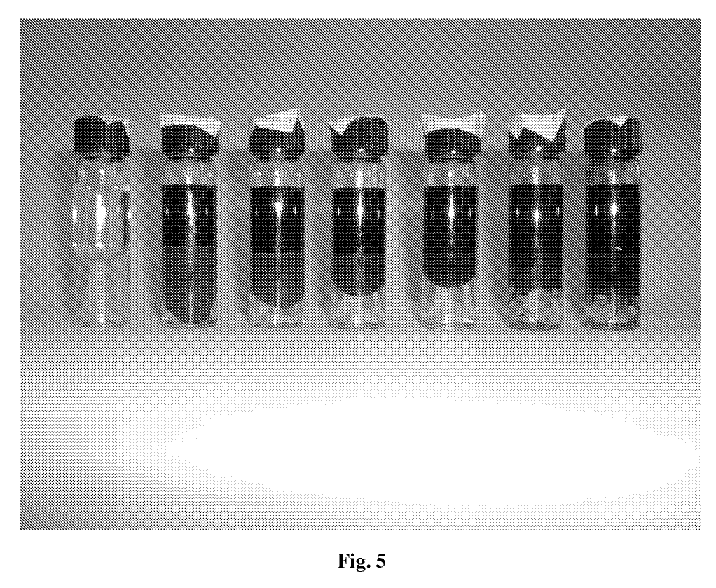

Different portions of the toluene soluble subfraction of asphaltenes from a preparative solubility sub-fractionation separation of n-heptane asphaltenes form Lloydminster vacuum residuum were added to 9-mL vial and suspended in 3.5 mL toluene. The suspensions were mechanically shaken until all of the asphaltenes were dissolved, 3.5 mL distilled water were added to the solution, and then these were agitated using a vortex mixture for 60 seconds. The results are shown in FIG. 5 after sitting at ambient temperature for 5 days. The blank toluene/water mixture is on the far left. From left to right, the various portions of the toluene soluble asphaltene Fraction 3 material added are: 5.0 mg, 10.5 mg, 21.0 mg, 42.0 mg, 85.0 mg, and 170.0 mg. It appears that the emulsion on the left, in which the smallest amount of the asphaltene subfraction was added, is an oil-in-water emulsion. At 42 mg, there is a catastrophic phase inversion from oil-in-water to water-in-oil. For the last 170 mg sample, it is possibly a mixed oil-in-water and water-in-oil emulsion. These results show that the type of emulsion (oil-in-water vs. water-in-oil) can be controlled by adding various amounts of asphaltenes or asphaltene subfractions materials.

### Emulsion Destabilization Using Sorbents

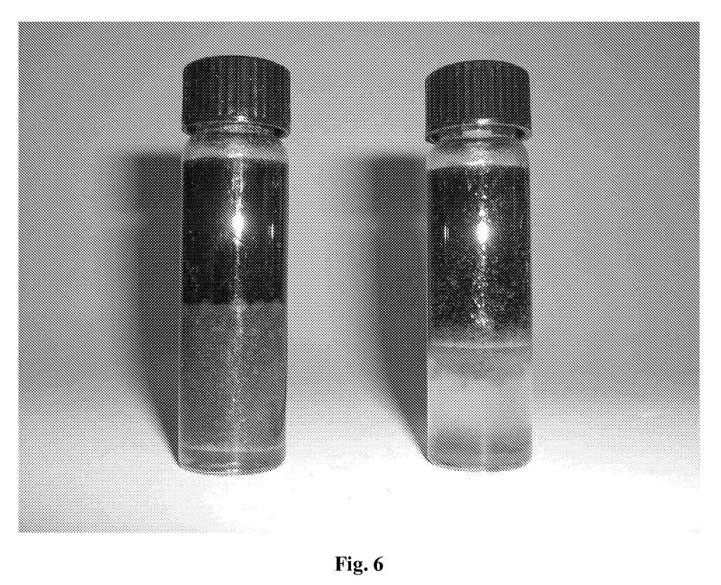

Portions of the most polar and pericondensed material form Lloydminster asphaltenes was added to two toluene and water mixtures in 9-mL vials. This methylene chloride soluble Fraction 4 asphaltene subfraction material was obtained form a preparative Asphaltene Determinator separating of Lloydminster vacuum residuum asphaltenes as described in Schabron et al. (2010). This material represents about 1.75% of the vacuum residuum oil. The two identical mixtures consisted of 4.5 mL of distilled water, 3.5 mL of toluene, and about 13 mg of the Fraction 4 material. They were then shaken for about 30 seconds to form partial emulsions. Stable emulsions were formed. The mixture on the left in FIG. 6 is the stable emulsion in the bottom water layer. About 100 mg of activated silica gel Grade 62 was added to the mixture in the vial on the right in FIG. 4. After being shaken briefly, the emulsion was broken, and clear water was evident in the bottom layer.

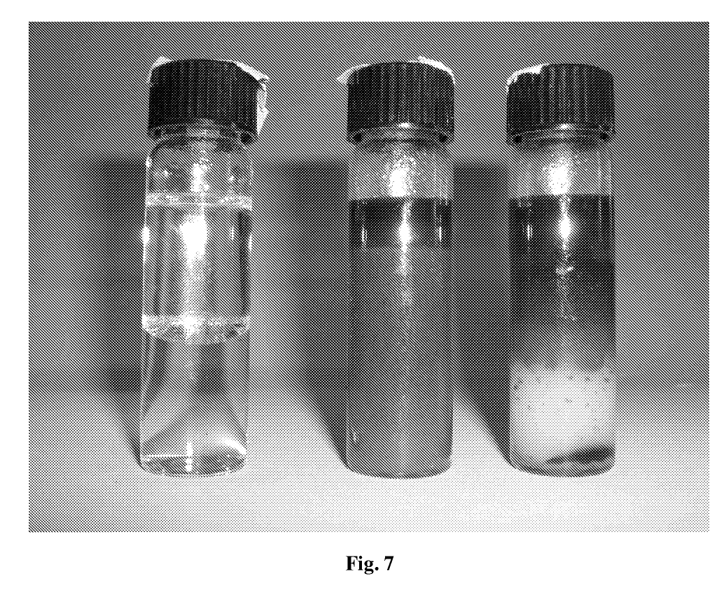

Results of a similar experiment with a different batch of methylene chloride soluble Fraction 4 asphaltene material from Lloydminster vacuum residuumn asphaltenes are shown in FIG. 7. The two identical mixtures consisted of 3.5 mL of distilled water, 3.5 mL of toluene, and about 5.1 mg of the Fraction 4 material. They were then agitated in a vortex mixture for about 60 seconds to form partial emulsions. Stable emulsions were formed. The mixture on the left in FIG. 7 is the stable emulsion in the bottom water layer. About 107 mg of activated silica gel Grade 62 was added to the mixture in the vial on the right in FIG. 7. After being shaken briefly, the emulsion was broken, and clear water was evident in the bottom layer. These results illustrate the ability of a sorbent to destabilize emulsions by removing some of the most aromatic (perhaps also the most polar and pericondensed) material from the system, possibly from the water droplet walls.

TABLE 1
Asphaltene Determinator Characterization of Light Desalter Inlet Oil.

| Sample: | WRI 1338-82-4 | | | Date: Mar. 15, 2011 | | | | | |
|---|---|---|---|---|---|---|---|---|---|
| | | Desalter Inlet Light Oil | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 69.52 | ELSD | 98.95 | 0.47 | 0.43 | 0.14 | 3.4 | | 2.0 |
| 2.0016 mg | | 500 nm | 47.99 | 14.08 | 26.23 | 11.71 | 1.2 | 0.55 | |
| | | 700 nm | 32.42 | 18.18 | 28.56 | 20.85 | 0.9 | | |

| | Whole Oil ELSD Area: 2016104 | | | | QC ELSD Area: | 6615140 | | |
|---|---|---|---|---|---|---|---|---|
| Corrected for | ELSD | 99.68 | 0.14 | 0.13 | 0.04 | 3.4 | | 0.6 |
| Volatiles Loss | 500 nm | 47.99 | 14.08 | 26.23 | 11.71 | 1.2 | 0.55 | |
| | 700 nm | 32.42 | 18.18 | 28.56 | 20.85 | 0.9 | | |

| | | Gravimetric Asphaltenes Analysis | | | | | Area Percent | Wt. % | Total |
|---|---|---|---|---|---|---|---|---|---|
| Material/ | Wt. % of | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 0.05 | ELSD | 10.52 | 23.22 | 61.78 | 4.49 | 66.27 | 0.03 | 0.11 |
| 10μ | | 500 nm | 6.68 | 25.99 | 61.23 | 6.11 | 67.34 | | |
| ~0.6800 mg | | 700 nm | 4.44 | 26.38 | 62.46 | 6.72 | 69.18 | | |
| C7 Asphaltenes | 0.06 | ELSD | 33.96 | 26.60 | 37.04 | 2.40 | 39.44 | 0.02 | |
| 0.45μ | | 500 nm | 14.50 | 35.48 | 45.88 | 4.14 | 50.02 | | |
| ~0.8400 mg | | 700 nm | 10.94 | 37.21 | 46.97 | 4.88 | 51.85 | | |

TABLE 2
Asphaltene Determinator Characterization of Desalted Light Oil.

| Sample: | WRI 1338-82-3 | | | | | | | | |
|---|---|---|---|---|---|---|---|---|---|
| | | Desalted Light Oil | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 70.34 | ELSD | 99.01 | 0.41 | 0.41 | 0.17 | 2.4 | | 1.8 |
| 2.0000 mg | | 500 nm | 46.38 | 14.09 | 26.99 | 12.53 | 1.1 | 0.58 | |
| | | 700 nm | 28.72 | 17.66 | 30.68 | 22.94 | 0.8 | | |
| | | Whole Oil ELSD Area: 1961945 | | | | QC ELSD Area: | 6615140 | | |
| Corrected for | | ELSD | 99.71 | 0.12 | 0.12 | 0.05 | 2.4 | | 0.5 |
| Volatiles Loss | | 500 nm | 46.38 | 14.09 | 26.99 | 12.53 | 1.1 | 0.58 | |
| | | 700 nm | 28.72 | 17.66 | 30.68 | 22.94 | 0.8 | | |
| | | Gravimetric Asphaltenes Analysis | | | | | Area Percent | Wt. % | Total |
| Material/ | Wt. % of | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 0.07 | ELSD | 20.30 | 20.68 | 55.47 | 3.55 | 59.02 | 0.04 | 0.12 |
| 10μ | | 500 nm | 6.29 | 26.81 | 61.29 | 5.61 | 66.90 | | |
| ~0.9600 mg | | 700 nm | 4.07 | 26.82 | 63.10 | 6.01 | 69.11 | | |
| C7 Asphaltenes | 0.05 | ELSD | 43.78 | 21.83 | 31.89 | 2.50 | 34.39 | 0.02 | |
| 0.45μ | | 500 nm | 16.46 | 32.84 | 45.66 | 5.04 | 50.70 | | |
| ~0.6800 mg | | 700 nm | 12.66 | 34.30 | 47.07 | 5.97 | 53.04 | | |

TABLE 3
Asphaltene Determinator Characterization of Light Oil Desalter Whole Emulsion, Shaken.

| Sample: | WRI 1338-82-5 | | | | | | | | |
|---|---|---|---|---|---|---|---|---|---|
| | | Light Oil Deesalter Emulsion with 40 vol. % Oil, Shaken | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 90.66 | ELSD | 97.67 | 1.34 | 0.62 | 0.37 | 3.6 | | na |
| 2.0018 mg | | 500 nm | Interference due to water turbidity for CH₂Cl₂:MeOH peak? | | | | | na | |
| | | 700 nm | Interference due to water turbidity for CH₂Cl₂:MeOH peak? | | | | | | |
| | | Whole Oil ELSD Area: 617842 | | | | QC ELSD Area: | 6615140 | | |
| Corrected for | | ELSD | 99.78 | 0.13 | 0.06 | 0.03 | 3.6 | | na |
| Volatiles Loss | | 500 nm | na | na | na | na | na | na | |
| | | 700 nm | na | na | na | na | na | | |
| | | Gravimetric Asphaltenes Analysis | | | | | Area Percent | Wt. % | Total |
| Material/ | Wt. % of | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 0.04 | ELSD | 17.92 | 18.35 | 57.11 | 6.62 | 63.73 | 0.03 | 0.11 |
| 10μ | | 500 nm | 6.40 | 22.00 | 62.24 | 9.37 | 71.61 | | |
| ~0.5600 mg | | 700 nm | 4.30 | 22.00 | 63.17 | 10.53 | 73.70 | | |
| C7 Asphaltenes | 0.07 | ELSD | 28.55 | 25.55 | 42.86 | 3.05 | 45.91 | 0.03 | |
| 0.45μ | | 500 nm | 12.47 | 32.79 | 49.71 | 5.03 | 54.74 | | |
| ~1.0000 mg | | 700 nm | 8.90 | 33.78 | 51.35 | 5.97 | 57.32 | | |

TABLE 4
Summary Results for the 500 nm Methylene Chloride:methanol
Peak Areas for the Light Oil Series.
Asphaltene Determinator CH₂Cl₂:MeOH (98:2 v:v) Area Percent

500 nm Relative Area Percent

| Light Crude API ^{~}40 | | 10 Micron |
|---|---|---|
| Material | Whole Oil | Asphaltenes |
| Inlet Crude | 11.71 | 6.11 |
| Rag layer Emulsion | na | 9.37 |
| Centrifuged Rag Layer Oil | na | 31.36 |
| Desalted Outlet Crude | 12.53 | 5.61 |

TABLE 5
Asphaltene Determinator Characterization of Light Supernatant Centrifuged Desalter Emulsion Oil.

| Sample: | WRI 1338-82-5 | | | | | | | | |
|---|---|---|---|---|---|---|---|---|---|
| | | Light Oil from Emulsion Separated by Centrifugation | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 68.21 | ELSD | 97.95 | 1.10 | 0.25 | 0.70 | 1.6 | | na |
| 2.0016 mg | | 500 nm | Interference due to water turbidity for CH₂Cl₂:MeOH peak? | | | | | na | |
| | | 700 nm | Interference due to water turbidity for CH₂Cl₂:MeOH peak? | | | | | | |
| | | Whole Oil ELSD Area: 2026156 | | | | QC ELSD Area: | 6373174.0 | | |
| Corrected for | | ELSD | 99.35 | 1.10 | 0.25 | 0.70 | 1.6 | | na |
| Volatiles Loss | | 500 nm | na | na | na | na | na | na | |
| | | 700 nm | na | na | na | na | na | | |
| | | Gravimetric Asphaltenes Analysis | | | | | Area Percent | Wt. % | Total |
| Material/ | Wt. % of | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 1.93 | ELSD | 12.58 | 28.32 | 35.61 | 23.48 | 59.09 | 1.14 | 2.32 |
| 10μ (dificult | | 500 nm | 5.00 | 15.02 | 48.63 | 31.36 | 79.99 | | |
| to filter) | | | | | | | | | |
| 0.4007 mg | | 700 nm | na | 14.32 | 45.70 | 39.98 | 85.68 | | |
| C7 Asphaltenes | 0.39 | ELSD | 63.05 | 11.26 | 17.66 | 8.03 | 25.69 | 0.10 | |
| 0.45μ | | 500 nm | 14.18 | 22.66 | 46.18 | 16.97 | 63.15 | | |
| 0.4051 mg | | 700 nm | 9.60 | 22.71 | 44.54 | 23.15 | 67.69 | | |

TABLE 6
Asphaltene Determinator Characterization of Medium Desalter Feed Oils
from Sets 1 and 2.

| Sample: | WRI 1338-94-21 | | | | | | | | |
|---|---|---|---|---|---|---|---|---|---|
| | | Desalter Inlet Medium Crude Set 1 | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 54.33 | ELSD | 95.26 | 1.44 | 2.81 | 0.49 | 2.9 | | 7.7 |
| 2.0012 mg | | 500 nm | 38.64 | 18.00 | 34.34 | 9.02 | 2.0 | 0.89 | |
| | | 700 nm | 24.32 | 21.59 | 38.85 | 15.24 | 1.4 | | |
| | | Whole Oil ELSD Area: 3096016 | | | | QC ELSD Area: | 6779303 | | |
| Corrected for | | ELSD | 97.84 | 0.66 | 1.28 | 0.22 | 2.9 | | 3.5 |
| Volatiles Loss | | 500 nm | 38.64 | 18.00 | 34.34 | 9.02 | 2.0 | 0.89 | |
| | | 700 nm | 24.32 | 21.59 | 38.85 | 15.24 | 1.4 | | |
| | | Gravimetric Asphaltenes Analysis | | | | | Area Percent | Wt. % | Total |
| Material/ | Wt. % of | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 4.27 | ELSD | 18.53 | 25.10 | 52.81 | 3.56 | 56.37 | 2.41 | 4.87 |
| 10μ | | 500 nm | 11.68 | 26.19 | 55.91 | 6.21 | 62.12 | | |
| 0.4010 mg | | 700 nm | 6.48 | 26.94 | 58.44 | 8.14 | 66.58 | | |
| C7 Asphaltenes | 0.60 | ELSD | 22.94 | 25.25 | 47.72 | 4.10 | 51.82 | 0.31 | |
| 0.45μ | | 500 nm | 12.86 | 27.55 | 53.33 | 6.26 | 59.59 | | |
| 0.4039 mg | | 700 nm | 8.53 | 28.16 | 55.36 | 7.95 | 63.31 | | |
| Sample: | WRI 1338-94-22 | | | | | | | | |
| | | Desalter Inlet Medium Crude Set 2 | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 54.87 | ELSD | 95.33 | 1.42 | 2.81 | 0.44 | 3.2 | | 7.6 |
| 2.0014 mg | | 500 nm | 38.55 | 18.06 | 34.38 | 9.01 | 2.0 | 0.89 | |
| Apr. 15, 2011 | | 700 nm | 23.93 | 21.51 | 39.18 | 15.38 | 1.4 | | |
| | | Whole Oil ELSD Area: 3059784 | | | | QC ELSD Area: | 6779303 | | |
| Corrected for | | ELSD | 97.89 | 0.64 | 1.27 | 0.20 | 3.2 | | 3.4 |
| Volatiles Loss | | 500 nm | 38.55 | 18.06 | 34.38 | 9.01 | 2.0 | 0.89 | |
| | | 700 nm | 23.93 | 21.51 | 39.18 | 15.38 | 1.4 | | |
| | | Gravimetric Asphaltenes Analysis | | | | | Area Percent | Wt. % | Total |
| Material/ | Wt. % of | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 4.32 | ELSD | 19.02 | 25.81 | 51.75 | 3.41 | 55.16 | 2.38 | 4.82 |
| 10μ | | 500 nm | 11.94 | 27.16 | 54.78 | 6.12 | 60.90 | | |
| 0.4008 mg | | 700 nm | 6.45 | 28.07 | 57.50 | 7.98 | 65.48 | | |
| C7 Asphaltenes | 0.50 | ELSD | 20.46 | 24.31 | 47.97 | 7.25 | 55.22 | 0.28 | |
| 0.45μ | | 500 nm | 13.49 | 26.11 | 52.51 | 7.89 | 60.40 | | |
| 0.4076 mg | | 700 nm | 6.74 | 26.84 | 55.43 | 10.99 | 66.42 | | |

TABLE 7
Asphaltene Determinator Characterization of Medium Desalted Outlet Oils
from Sets 1 and 2.

| Sample: | WRI 1338-94-23 | | | | | | | | |
|---|---|---|---|---|---|---|---|---|---|
| | | Desalter Inlet Medium Crude Set 1 | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 53.58 | ELSD | 95.27 | 1.49 | 2.78 | 0.45 | 3.3 | | 7.8 |
| 2.0002 mg | | 500 nm | 39.06 | 18.50 | 33.77 | 8.67 | 2.1 | 0.86 | |
| | | 700 nm | 24.16 | 21.99 | 38.53 | 15.32 | 1.4 | | |
| | | Whole Oil ELSD Area: 3147093 | | | | QC ELSD Area: | 6779303 | | |
| Corrected for | | ELSD | 97.80 | 0.69 | 1.29 | 0.21 | 3.3 | | 3.6 |
| Volatiles Loss | | 500 nm | 39.06 | 18.50 | 33.77 | 8.67 | 2.1 | 0.86 | |
| | | 700 nm | 24.16 | 21.99 | 38.53 | 15.32 | 1.4 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 4.22 | ELSD | 20.42 | 26.30 | 50.09 | 3.20 | 53.29 | 2.25 | 4.97 |
| 10μ | | 500 nm | 11.99 | 27.51 | 54.49 | 6.01 | 60.50 | | |
| 0.4004 mg | | 700 nm | 6.49 | 28.42 | 57.27 | 7.82 | 65.09 | | |
| C7 Asphaltenes | 0.75 | ELSD | 28.76 | 21.24 | 45.82 | 4.18 | 50.00 | 0.38 | |
| 0.45μ | | 500 nm | 13.01 | 24.80 | 55.31 | 6.87 | 62.18 | | |
| 0.4019 mg | | 700 nm | 7.42 | 25.43 | 58.17 | 8.98 | 67.15 | | |
| Sample: | WRI 1338-94-24 | | | | | | | | |
| | | Desalted Medium Crude Set 2 | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 53.40 | ELSD | 95.60 | 1.47 | 2.48 | 0.45 | 3.3 | | 7.4 |
| 2.0006 mg | | 500 nm | 40.90 | 19.91 | 31.76 | 7.42 | 2.7 | 0.78 | |
| | | 700 nm | 26.69 | 24.00 | 37.83 | 11.48 | 2.1 | | |
| | | Whole Oil ELSD Area: 2723177 | | | | QC ELSD Area: | 5843181 | | |
| Corrected for | | ELSD | 97.95 | 0.69 | 1.16 | 0.21 | 3.3 | | 3.5 |
| Volatiles Loss | | 500 nm | 40.90 | 19.91 | 31.76 | 7.42 | 2.7 | 0.78 | |
| | | 700 nm | 26.69 | 24.00 | 37.83 | 11.48 | 2.1 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 2.27 | ELSD | 10.93 | 23.33 | 62.27 | 3.46 | 65.73 | 1.49 | 2.6 |
| 10μ | | 500 nm | 8.51 | 24.60 | 61.87 | 5.03 | 66.90 | | |
| 0.4002 mg | | 700 nm | 4.78 | 24.89 | 64.05 | 6.28 | 70.33 | | |
| C7 Asphaltenes | 0.31 | ELSD | 23.69 | 21.36 | 51.98 | 2.97 | 54.95 | 0.17 | |
| 0.45μ | | 500 nm | 11.10 | 25.55 | 58.25 | 5.10 | 63.35 | | |
| 0.4120 mg | | 700 nm | 6.46 | 26.31 | 60.68 | 6.55 | 67.23 | | |

TABLE 8
Asphaltene Determinator Characterization of Medium Oil Desalter Emulsions
from Sets 1 and 2.

| Sample: | WRI 1338-94-25 | | | | | | | | |
|---|---|---|---|---|---|---|---|---|---|
| | | Medium Crude Emulsion Shaken (Contains 60 vol. % Oil) Set 1 | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 89.38 | ELSD | 93.87 | 1.61 | 3.21 | 1.31 | 1.2 | | 8.7 |
| 2.0000 mg | | 500 nm | 29.28 | 17.15 | 39.90 | 13.67 | 1.3 | 1.36 | |
| | | 700 nm | 14.85 | 18.09 | 45.02 | 22.04 | 0.8 | | |
| | | Whole Oil ELSD Area: 620678 | | | | QC ELSD Area: | 5843181 | | |
| Corrected for | | ELSD | 99.35 | 0.17 | 0.34 | 0.14 | 1.2 | | 0.9 |
| Volatiles Loss | | 500 nm | 29.28 | 17.15 | 39.90 | 13.67 | 1.3 | 1.36 | |
| | | 700 nm | 14.85 | 18.09 | 45.02 | 22.04 | 0.8 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 1.62 | ELSD | 13.04 | 18.00 | 58.62 | 10.34 | 68.96 | 1.12 | 1.76 |
| 10μ | | 500 nm | 7.09 | 18.90 | 62.39 | 11.62 | 74.01 | | |
| 0.4075 mg | | 700 nm | 3.90 | 17.92 | 63.30 | 14.88 | 78.18 | | |
| C7 Asphaltenes | 0.14 | ELSD | 30.96 | 23.48 | 39.62 | 5.93 | 45.55 | 0.06 | |
| 0.45μ | | 500 nm | 14.89 | 26.44 | 44.84 | 13.83 | 58.67 | | |
| 0.3900 mg | | 700 nm | 7.93 | 24.62 | 42.63 | 24.82 | 67.45 | | |
| Sample: | WRI 1338-94-26 | | | | | | | | |
| | | Medium Crude Emulsion Shaken (Contains 80 vol. % Oil) Set 2 | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 89.76 | ELSD | 94.06 | 1.57 | 3.02 | 1.35 | 1.2 | | 8.5 |
| 2.0002 mg | | 500 nm | 29.99 | 17.49 | 39.48 | 13.04 | 1.3 | 1.32 | |
| | | 700 nm | 19.13 | 19.43 | 41.43 | 20.00 | 1.0 | | |
| | | Whole Oil ELSD Area: 598385 | | | | QC ELSD Area: | 5843181 | | |
| Corrected for | | ELSD | 99.39 | 0.16 | 0.31 | 0.14 | 1.2 | | 0.9 |
| Volatiles Loss | | 500 nm | 29.99 | 17.49 | 39.48 | 13.04 | 1.3 | 1.32 | |
| | | 700 nm | 19.13 | 19.43 | 41.43 | 20.00 | 1.0 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 2.16 | ELSD | 10.81 | 15.05 | 48.61 | 25.54 | 74.15 | 1.60 | 2.47 |
| 10μ | | 500 nm | 5.66 | 12.59 | 44.56 | 37.19 | 81.75 | | |
| 0.4004 mg | | 700 nm | 2.69 | 9.01 | 34.39 | 53.91 | 88.30 | | |
| C7 Asphaltenes | 0.31 | ELSD | 14.73 | 20.01 | 47.44 | 17.81 | 65.25 | 0.20 | |
| 0.45μ | | 500 nm | 7.70 | 16.93 | 41.97 | 33.40 | 75.37 | | |
| 0.4160 mg | | 700 nm | 3.50 | 12.89 | 32.36 | 51.25 | 83.61 | | |

TABLE 9
Asphaltene Determinator Characterization of Medium Desalter Centrifuged Oils
from Emulsions from Sets 1 and 2.

| Sample: | WRI 1338-94-25 | | | | | | | | |
|---|---|---|---|---|---|---|---|---|---|
| | | Medium Crude Emulsion Oil Separated by Centrifugation Set 1 | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 56.51 | ELSD | 94.70 | 1.89 | 2.93 | 0.48 | 3.9 | | 8.2 |
| 2.0005 mg | | 500 nm | 35.22 | 20.13 | 35.04 | 9.61 | 2.1 | 0.99 | |
| | | 700 nm | 22.71 | 23.39 | 39.16 | 14.74 | 1.6 | | |
| | | Whole Oil ELSD Area: 2979118 | | | | QC ELSD Area: | 6849664 | | |
| Corrected for | | ELSD | 97.70 | 0.82 | 1.27 | 0.21 | 3.9 | | 3.6 |
| Volatiles Loss | | 500 nm | 35.22 | 20.13 | 35.04 | 9.61 | 2.1 | 0.99 | |
| | | 700 nm | 22.71 | 23.39 | 39.16 | 14.74 | 1.6 | | |
| | | Gravimetric Asphaltenes Analysis | | | | | Area Percent | Wt. % | Total |
| Material/ | Wt. % of | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 1.84 | ELSD | 14.87 | 30.22 | 51.76 | 3.15 | 54.91 | 1.01 | 2.69 |
| 10μ | | 500 nm | 10.67 | 31.85 | 52.36 | 5.12 | 57.48 | | |
| 0.3981 mg | | 700 nm | 6.37 | 32.94 | 54.26 | 6.43 | 60.69 | | |
| C7 Asphaltenes | 0.85 | ELSD | 27.25 | 26.10 | 43.67 | 2.98 | 46.65 | 0.40 | |
| 0.45μ | | 500 nm | 12.35 | 31.39 | 51.06 | 5.20 | 56.26 | | |
| 0.4240 mg | | 700 nm | 7.41 | 32.65 | 53.25 | 6.69 | 59.94 | | |
| Sample: | WRI 1338-94-26 | | | | | | | | |
| | | Medium Crude Emulsion Oil Separated by Centrifugation Set 2 | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 56.52 | ELSD | 94.92 | 1.88 | 2.78 | 0.42 | 4.5 | | 7.9 |
| 2.0006 mg | | 500 nm | 35.92 | 20.14 | 35.01 | 8.93 | 2.3 | 0.97 | |
| | | 700 nm | 22.77 | 23.54 | 39.58 | 14.11 | 1.7 | | |
| | | Whole Oil ELSD Area: 2978183 | | | | QC ELSD Area: | 6849664 | | |
| Corrected for | | ELSD | 97.79 | 0.82 | 1.21 | 0.18 | 4.5 | | 3.4 |
| Volatiles Loss | | 500 nm | 35.92 | 20.14 | 35.01 | 8.93 | 2.3 | 0.97 | |
| | | 700 nm | 22.77 | 23.54 | 39.58 | 14.11 | 1.7 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 1.96 | ELSD | 15.30 | 29.18 | 50.56 | 4.96 | 55.52 | 1.09 | 2.81 |
| 10μ | | 500 nm | 11.29 | 30.98 | 51.69 | 6.04 | 57.73 | | |
| 0.4008 mg | | 700 nm | 6.80 | 31.86 | 53.39 | 7.94 | 61.33 | | |
| C7 Asphaltenes | 0.85 | ELSD | 21.55 | 28.34 | 46.90 | 3.21 | 50.11 | 0.43 | |
| 0.45μ | | 500 nm | 12.45 | 31.34 | 50.92 | 5.30 | 56.22 | | |
| 0.4012 mg | | 700 nm | 7.27 | 32.67 | 53.43 | 6.63 | 60.06 | | |

TABLE 10
Summary of Results for the 500 nm Methylene Chloride:methanol
Peak Areas for the Medium Oil Sets 1and 2.
Asphaltene Determinator CH₂Cl₂:MeOH (98:2 v:v) Area Percent

500 nm Relative Area Percent

| | | 10 Micron |
|---|---|---|
| Material | Whole Oil | Asphaltenes |
| Medium Crude 1 API ^{~}25 | | |
| Inlet Crude | 9.02 | 6.21 |
| Rag layer Emulsion | 13.67 | 11.62 |
| Centrifuged Rag Layer Oil | 9.61 | 5.12 |
| Desalted Outlet Crude | 8.67 | 6.01 |
| Medium Crude 2 API ^{~}25 | | |
| Inlet Crude | 9.01 | 6.12 |
| Rag layer Emulsion | 13.04 | 37.19 |
| Centrifuged Rag Layer Oil | 8.93 | 6.04 |
| Desalted Outlet Crude | 7.42 | 5.03 |

TABLE 11
Amount of Oil, Water, and Sediment following
Centrifugation of Emulsions.
Emulsions from Heavy Crude Set (API ^{~}20)

| | Centrifuged Volume Percent | | |
|---|---|---|---|
| Try Layer | Oil | Water | Sediment |
| T-1 | 50 | 34 | 16 |
| T-2 | 40 | 49 | 11 |
| T-3 | 56 | 30 | 14 |
| T-4 | 56 | 35 | 9 |

TABLE 12
Relative 500 nm Absorbance Detector Peak Area Percents for the Most
Pericondensed Material in the Heavy Oil Desalter Sample Set.
Asphaltene Determinator 500 nm CH₂Cl₂:MeOH (98:2 v:v) Peaks

| | 500 nm Relative Area Percent | |
|---|---|---|
| | Whole | 10μ |
| Heavy Crude API ~20 | Material | Asphaltenes |

Sample

| | | |
|---|---|---|
| Inlet Crude | 4.31 | 3.03 |
| Desalted Outlet Crude | 4.12 | 2.86 |

Try Layer 1

| | | |
|---|---|---|
| Whole Sample Shaken | 5.74 | 11.23 |
| Whole Emulsion | 5.84 | 10.58 |
| Emulsion Centrifuged Oil | 3.73 | 3.91 |
| Emulsion Centrifuged Water, Evaporated | 7.71 | na |

Try Layer 2

| | | |
|---|---|---|
| Whole Sample Shaken | 5.37 | 13.71 |
| Whole Emulsion | 5.77 | 10.99 |
| Emulsion Centrifuged Oil | 3.95 | 3.94 |
| Emulsion Centrifuged Water, Evaporated | 6.82 | na |
| Bottle 2 Centrifuged Emulsion | 18.79 | na |
| Water, Evaporated | | |

Try Layer 3

| | | |
|---|---|---|
| Whole Sample Shaken | 5.69 | 13.99 |
| Top Layer Oil Only | 4.69 | 3.70 |
| Whole Emulsion | 5.74 | 13.08 |
| Emulsion Centrifuged Oil | 5.79 | 4.13 |
| Emulsion Centrifuged Water, Evaporated | 4.48 | na |
| Middle Bottle 2 Whole Emulsion | 29.03 | 4.65 |
| Middle Bottle 2 Water, Evaporated | 8.16 | na |

Try Layer 4

| | | |
|---|---|---|
| Whole Sample Shaken | 5.45 | 8.74 |
| Whole Emulsion | 5.29 | 3.75 |
| Emulsion Centrifuged Oil | 4.00 | 3.75 |
| Emulsion Centrifuged Water, Evaporated | 3.39 | na |

Try Layer 5

| | | |
|---|---|---|
| Whole Oil | 4.13 | 4.14 |

Try Layer 7

| | | |
|---|---|---|
| Whole Oil | 3.93 | 4.14 |

TABLE 13
Relative Volatiles Corrected ELSD Peak Area Percents for the Most
Pericondensed Material in the Heavy Oil Desalter Sample Set.
Asphaltene Determinater Volatiles Corrected ELSD Peaks

| | | CH₂Cl₂:MeOH | |
|---|---|---|---|
| | | (98:2 v:v) Peak | |
| | Volatiles | ELSD Relative | |
| | Corrected | Area Percent | |
| | Area % C7 | Whole | 10μ |
| Heavy Crude API ^{~}20 | Insolubles | Material | Asphaltenes |

Sample

| | | | |
|---|---|---|---|
| Inlet Crude | 3.67 | 0.09 | 0.90 |
| Desalted Outlet Crude | 3.84 | 0.09 | 0.84 |

Try Layer 1

| | | | |
|---|---|---|---|
| Whole Sample Shaken | 1.91 | 0.08 | 5.79 |
| Whole Emulsion | 2.06 | 0.11 | 6.40 |
| Emulsion Centrifuged Oil | 3.58 | 0.13 | 2.52 |
| Emulsion Centrifuged Water, | 7.76 | 0.45 | na |
| Evaporated | | | |

Try Layer 2

| | | | |
|---|---|---|---|
| Whole Sample Shaken | 2.16 | 0.09 | 6.87 |
| Whole Emulsion | 2.19 | 0.10 | 5.90 |
| Emulsion Centrifuged Oil | 3.60 | 0.14 | 2.27 |
| Emulsion Centrifuged Water, | 7.18 | 0.57 | na |
| Evaporated | | | |
| Bottle 2 Centrifuged Emulsion | 31.99 | 2.20 | na |
| Water, Evaporated | | | |

Try Layer 3

| | | | |
|---|---|---|---|
| Whole Sample Shaken | 2.30 | 0.10 | 6.89 |
| Top Layer Oil Only | 1.96 | 0.20 | 2.14 |
| Whole Emulsion | 2.15 | 0.34 | 7.29 |
| Emulsion Centrifuged Oil | 2.55 | 0.15 | 2.46 |
| Emulsion Centrifuged Water, | 6.27 | 0.77 | na |
| Evaporated | | | |
| Middle Bottle 2 Whole Emulsion | 0.31 | 0.17 | 2.20 |
| Middle Bottle 2 Water, | 22.67 | 1.87 | na |
| Evaporated | | | |

Try Layer 4

| | | | |
|---|---|---|---|
| Whole Sample Shaken | 2.11 | 0.09 | 4.74 |
| Whole Emulsion | 2.60 | 0.10 | 1.82 |
| Emulsion Centrifuged Oil | 5.29 | 0.17 | 1.68 |
| Emulsion Centrifuged Water, | 8.94 | 0.79 | na |
| Evaporated | | | |

Try Layer 5

| | | | |
|---|---|---|---|
| Whole Oil | 3.97 | 0.11 | 1.11 |

Try Layer 7

| | | | |
|---|---|---|---|
| Whole Oil | 4.19 | 0.10 | 0.92 |

TABLE 14
Relative ELSD Non-Volatile Component ELSD Peak Area Percents for the
Most Polar and Pericondensed Material in the Heavy Oil Desalter Sample Set.
Asphaltene Determinator ELSD Peaks for ELSD non-Volatile Components

| | | | | CH₂Cl₂:MeOH | |
|---|---|---|---|---|---|
| | | | | (98:2 v:v) Peak | |
| | Area | ELSD Non- | | ELSD Relative | |
| | Percent | Volatile | | Area Percent | |
| | ELSD | Area % C7 | Percent | Whole | 10μ |
| Heavy Crude API ^{~}20 | Volatiles | Insolubles | TPA | Material | Asphaltenes |

Sample

| | | | | | |
|---|---|---|---|---|---|
| Inlet Crude | 41.02 | 6.22 | 9.8 | 0.16 | 0.90 |
| Desalted Outlet Crude | 40.85 | 6.49 | 10.2 | 0.16 | 0.84 |

Try Layer 1

| | | | | | |
|---|---|---|---|---|---|
| Whole Sample Shaken | 74.29 | 7.43 | 11.5 | 0.31 | 5.79 |
| Whole Emulsion | 68.58 | 7.19 | 11.6 | 0.35 | 6.40 |
| Emulsion Centrifuged Oil | 50.91 | 7.29 | 11.4 | 0.27 | 2.52 |
| Emulsion Centrifuged | 70.88 | 26.64 | 37.1 | 10.41 | na |
| Water, Evaporated | | | | | |

Try Layer 2

| | | | | | |
|---|---|---|---|---|---|
| Whole Sample Shaken | 69.82 | 7.17 | 11.1 | 0.29 | 6.87 |
| Whole Emulsion | 70.26 | 7.35 | 11.9 | 0.33 | 5.90 |
| Emulsion Centrifuged Oil | 51.69 | 7.45 | 11.6 | 0.28 | 2.27 |
| Emulsion Centrifuged | 79.21 | 34.53 | 51.3 | 11.84 | na |
| Water, Evaporated | | | | | |
| Bottle 2 Centrifuged Emulsion | 60.35 | 80.68 | 103.6 | 5.55 | na |
| Water, Evaporated | | | | | |

Try Layer 3

| | | | | | |
|---|---|---|---|---|---|
| Whole Sample Shaken | 69.39 | 7.52 | 11.7 | 0.34 | 6.89 |
| Top Layer Oil Only | 79.99 | 9.80 | 14.3 | 1.01 | 2.14 |
| Whole Emulsion | 69.47 | 7.05 | 11.5 | 0.90 | 7.29 |
| Emulsion Centrifuged Oil | 69.11 | 8.25 | 12.6 | 0.47 | 2.46 |
| Emulsion Centrifuged Water, | 56.33 | 14.35 | 22.9 | 0.77 | na |
| Evaporated | | | | | |
| Middle Bottle 2 Whole | 99.48 | 60.51 | 74.6 | 33.42 | 2.20 |
| Emulsion | | | | | |
| Middle Bottle 2 Water, | 58.54 | 54.68 | 84.4 | 1.87 | na |
| Evaporated | | | | | |

Try Layer 4

| | | | | | |
|---|---|---|---|---|---|
| Whole Sample Shaken | 73.87 | 8.06 | 12.1 | 0.36 | 4.74 |
| Whole Emulsion | 62.98 | 7.03 | 11.3 | 0.27 | 1.82 |
| Emulsion Centrifuged Oil | 33.04 | 7.90 | 12.4 | 0.25 | 1.68 |
| Emulsion Centrifuged | 48.87 | 17.48 | 29.0 | 3.38 | na |
| Water, Evaporated | | | | | |

Try Layer 5

| | | | | | |
|---|---|---|---|---|---|
| Whole Oil | 40.24 | 6.65 | 10.5 | 0.18 | 1.11 |

Try Layer 7

| | | | | | |
|---|---|---|---|---|---|
| Whole Oil | 37.91 | 6.75 | 10.5 | 0.16 | 0.92 |

TABLE 15
Relative 500 nm Absorbance Detector Peak Area Percents for the
Gravimetric Asphaltenes from the Heavy Oil Desalter Sample Set.
Asphaltene Determinator 500 nm Peaks
from 10μ Gravimetric Asphaltenes

| Asphaltenes from | | 500 nm Relative Area Percent | |
|---|---|---|---|
| Heavy Crude API ~20 | wt. % | Toluene | CH2Cl2:MeOH |

Sample

| | | | |
|---|---|---|---|
| Inlet Crude | 7.51 | 54.14 | 3.03 |
| Desalted Outlet Crude | 8.21 | 52.35 | 2.86 |

Try Layer 1

| | | | |
|---|---|---|---|
| Whole Oil | 2.86 | 51.94 | 11.23 |
| Whole Emulsion | 2.39 | 53.44 | 10.58 |
| Emulsoin Centrifuged Oil | 3.54 | 53.20 | 3.91 |

Try Layer 2

| | | | |
|---|---|---|---|
| Whole Oil | 1.79 | 53.13 | 13.71 |
| Whole Emulsion | 1.72 | 54.05 | 10.99 |
| Emulsoin Centrifuged Oil | 6.92 | 52.08 | 3.94 |

Try Layer 3

| | | | |
|---|---|---|---|
| Whole Oil | 2.95 | 52.78 | 13.99 |
| Top 1.5 Inch Oil | 0.78 | 57.17 | 3.70 |
| Whole Emulsion | 2.00 | 49.85 | 13.08 |
| Emulsoin Centrifuged Oil | 5.95 | 51.39 | 4.13 |

Try Layer 4

| | | | |
|---|---|---|---|
| Whole Oil | 3.29 | 55.37 | 8.74 |
| Whole Emulsion | 4.88 | 57.21 | 3.75 |
| Emulsoin Centrifuged Oil | 9.06 | 51.55 | 3.75 |

Try Layer 5

| | | | |
|---|---|---|---|
| Whole Oil | 8.97 | 52.63 | 4.14 |

Try Layer 7

| | | | |
|---|---|---|---|
| Whole Oil | 8.98 | 50.33 | 3.82 |

### APPENDIX A

### Asphaltene Determinator Data for Heavy Oil Desalter Emulsion Samples

| Sample: | WRI 1338-131-9 (#4 Raw) | | | | Heavy Oil Set 3 | | | | |
|---|---|---|---|---|---|---|---|---|---|
| | | Desalter Inlet Oil | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 41.02 | ELSD | 93.78 | 2.17 | 3.89 | 0.16 | 13.6 | | 9.8 |
| 2.0000 mg | | 500 nm | 36.39 | 23.64 | 35.66 | 4.31 | 5.5 | 0.98 | |
| | | 700 nm | 24.58 | 26.67 | 42.17 | 6.58 | 4.1 | | |
| | | Whole Oil ELSD Area: 4478821 | | | | QC ELSD Area: | 7594086 | | |
| Corrected for | | ELSD | 96.33 | 1.28 | 2.29 | 0.09 | 13.6 | | 5.8 |
| Volatiles Loss | | 500 nm | 36.39 | 23.64 | 35.66 | 4.31 | 5.5 | 0.98 | |
| | | 700 nm | 24.58 | 26.67 | 42.17 | 6.58 | 4.1 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 7.51 | ELSD | 13.34 | 25.76 | 60.00 | 0.90 | 60.90 | 4.57 | 7.54 |
| 10μ | | 500 nm | 13.87 | 28.96 | 54.14 | 3.03 | 57.17 | | |
| .4012 mg | | 700 nm | 9.16 | 30.22 | 56.90 | 3.72 | 60.62 | | |
| C7 Asphaltenes | 0.03 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-27A (2nd Desalted) | | | | Heavy Oil Set 3 | | | | |
| | | Desalted Outlet Oil | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 40.85 | ELSD | 93.51 | 2.31 | 4.01 | 0.16 | 14.4 | | 10.2 |
| 2.0000 mg | | 500 nm | 36.31 | 23.72 | 35.85 | 4.12 | 5.8 | 0.99 | |
| | | 700 nm | 24.61 | 26.80 | 42.47 | 6.12 | 4.4 | | |
| | | Whole Oil ELSD Area: 4492090 | | | | QC ELSD Area: | 7594086 | | |
| Corrected for | | ELSD | 96.16 | 1.37 | 2.37 | 0.09 | 14.4 | | 6.0 |
| Volatiles Loss | | 500 nm | 36.31 | 23.72 | 35.85 | 4.12 | 5.8 | 0.99 | |
| | | 700 nm | 24.61 | 26.80 | 42.47 | 6.12 | 4.4 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 8.21 | ELSD | 16.64 | 25.67 | 56.85 | 0.84 | 57.69 | 4.74 | 8.27 |
| 10μ | | 500 nm | 15.12 | 29.68 | 52.35 | 2.86 | 55.21 | | |
| .4002 mg | | 700 nm | 10.31 | 31.03 | 55.03 | 3.63 | 58.66 | | |
| C7 Asphaltenes | 0.06 | ELSD | 49.07 | 15.79 | 34.34 | 0.79 | 35.13 | 0.02 | |
| 0.45μ | | 500 nm | 23.04 | 26.17 | 46.33 | 4.46 | 50.79 | | |
| .2340 mg | | 700 nm | 17.97 | 26.95 | 48.97 | 6.11 | 55.08 | | |
| Sample: | WRI 1338-131-10 (#1st Try Layer-1) | | | | Heavy Oil Set 3 | | | | |
| | | Whole Sample Shaken | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 74.29 | ELSD | 92.57 | 2.80 | 4.32 | 0.31 | 9.0 | | 11.5 |
| 2.0008 mg | | 500 nm | 35.42 | 23.19 | 35.65 | 5.74 | 4.0 | 1.01 | |
| | | 700 nm | 24.37 | 26.23 | 41.09 | 8.32 | 3.2 | | |
| | | Whole Oil ELSD Area: 1952211 | | | | QC ELSD Area: | 7594086 | | |
| Corrected for | | ELSD | 98.09 | 0.72 | 1.11 | 0.08 | 9.0 | | 3.0 |
| Volatiles Loss | | 500 nm | 35.42 | 23.19 | 35.65 | 5.74 | 4.0 | 1.01 | |
| | | 700 nm | 24.37 | 26.23 | 41.09 | 8.32 | 3.2 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 2.86 | ELSD | 7.26 | 25.01 | 61.94 | 5.79 | 67.73 | 1.94 | 2.88 |
| 10μ | | 500 nm | 11.73 | 25.10 | 51.94 | 11.23 | 63.17 | | |
| 0.4014 mg | | 700 nm | 6.76 | 22.99 | 49.93 | 20.31 | 70.24 | | |
| C7 Asphaltenes | 0.02 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-10 (#1st Try Layer-1) | | | | Heavy Oil Set 3 | | | | |
| | | Whole Emulsion Layer Shaken | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 68.58 | ELSD | 92.81 | 2.34 | 4.49 | 0.35 | 6.7 | | 11.6 |
| 2.0016 mg | | 500 nm | 37.95 | 20.49 | 35.71 | 5.84 | 3.5 | 0.94 | |
| | | 700 nm | 26.36 | 22.72 | 41.86 | 9.06 | 2.5 | | |
| | | Whole Oil ELSD Area: 2532470 | | | | QC ELSD Area: | 8060927 | | |
| Corrected for | | ELSD | 97.74 | 0.74 | 1.41 | 0.11 | 6.7 | | 3.6 |
| Volatiles Loss | | 500 nm | 37.95 | 20.49 | 35.71 | 5.84 | 3.5 | 0.94 | |
| | | 700 nm | 26.36 | 22.72 | 41.86 | 9.06 | 2.5 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 2.39 | ELSD | 8.50 | 22.37 | 62.73 | 6.40 | 69.13 | 1.35 | 2.40 |
| 10μ | | 500 nm | 12.07 | 23.90 | 53.44 | 10.58 | 64.02 | | |
| 0.4004 mg | | 700 nm | 8.23 | 24.04 | 55.08 | 12.64 | 67.72 | | |
| C7 Asphaltenes | 0.01 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-10 (#1st T-1) | | | | Heavy Oil Set 3 | | | | |
| | | Centrifuged Oil from Emulsion Layer | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 50.91 | ELSD | 92.71 | 1.93 | 5.08 | 0.27 | 7.1 | | 11.4 |
| 2.0016 mg | | 500 nm | 36.02 | 23.02 | 37.23 | 3.73 | 6.2 | 1.03 | |
| | | 700 nm | 25.44 | 25.91 | 42.97 | 5.68 | 4.6 | | |
| | | Whole Oil ELSD Area: 3850429 | | | | QC ELSD Area: | 7842919 | | |
| Corrected for | | ELSD | 96.42 | 0.95 | 2.49 | 0.13 | 7.1 | | 5.6 |
| Volatiles Loss | | 500 nm | 36.02 | 23.02 | 37.23 | 3.73 | 6.2 | 1.03 | |
| | | 700 nm | 25.44 | 25.91 | 42.97 | 5.68 | 4.6 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 3.54 | ELSD | 11.12 | 22.17 | 64.19 | 2.52 | 66.71 | 2.37 | 3.55 |
| 10μ | | 500 nm | 14.51 | 28.38 | 53.20 | 3.91 | 57.11 | | |
| 0.4020 mg | | 700 nm | 9.68 | 28.94 | 56.19 | 5.18 | 61.37 | | |
| C7 Asphaltenes | 0.01 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-10 (#1st Try Layer-1) | | | | Heavy Oil Set 3 | | | | |
| | | Oil Residue from Evaporated Centrifuged Water Fraction from Emulsion Layer | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 70.88 | ELSD | 73.36 | 3.02 | 13.20 | 10.41 | 0.3 | | 37.1 |
| 0.2947 mg | | 500 nm | 28.26 | 24.03 | 40.01 | 7.71 | 3.1 | 1.42 | |
| | | 700 nm | 23.00 | 25.75 | 40.50 | 10.76 | 2.4 | | |
| | | Whole Oil 2 mg ELSD Area: 2283936 | | | | QC ELSD Area: | 7842919 | | |
| Corrected for | | ELSD | 92.24 | 0.88 | 3.84 | 3.03 | 0.3 | | |
| Volatiles Loss | | 500 nm | 28.26 | 24.03 | 40.01 | 7.71 | 3.1 | | |
| | | 700 nm | 23.00 | 25.75 | 40.50 | 10.76 | 2.4 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | | ELSD | Insufficient Material | | | | | | |
| 10μ | | 500 nm | Insufficient Material | | | | | | |
| | | 700 nm | Insufficient Material | | | | | | |
| C7 Asphaltenes | | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-11 (#1st Try Layer-2) | | | | Heavy Oil Set 3 | | | | |
| | | Whole Sample Shaken | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 69.82 | ELSD | 92.83 | 2.50 | 4.38 | 0.29 | 8.6 | | 11.1 |
| 2.0004 mg | | 500 nm | 35.49 | 23.13 | 36.01 | 5.37 | 4.3 | 1.01 | |
| | | 700 nm | 24.04 | 26.30 | 42.04 | 7.62 | 3.5 | | |
| | | Whole Oil ELSD Area: 2291864 | | | | QC ELSD Area: | 7594086 | | |
| Corrected for | | ELSD | 97.84 | 0.75 | 1.32 | 0.09 | 8.6 | | 3.4 |
| Volatiles Loss | | 500 nm | 35.49 | 23.13 | 36.01 | 5.37 | 4.3 | 1.01 | |
| | | 700 nm | 24.04 | 26.30 | 42.04 | 7.62 | 3.5 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 1.79 | ELSD | 4.48 | 23.95 | 64.70 | 6.87 | 71.57 | 1.28 | 1.80 |
| 10μ | | 500 nm | 9.62 | 23.54 | 53.13 | 13.71 | 66.84 | | |
| 0.4028 mg | | 700 nm | 5.60 | 21.34 | 49.71 | 23.34 | 73.05 | | |
| C7 Asphaltenes | 0.01 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-11(#1st Try Layer-2) | | | | Heavy Oil Set 3 | | | | |
| | | Whole Emulsion Layer Shaken | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 70.26 | ELSD | 92.65 | 2.42 | 4.59 | 0.33 | 7.3 | | 11.9 |
| 2.0000 mg | | 500 nm | 38.03 | 20.73 | 35.48 | 5.77 | 3.6 | 0.93 | |
| | | 700 nm | 26.80 | 22.67 | 40.73 | 9.80 | 2.3 | | |
| | | Whole Oil ELSD Area: 2397115 | | | | QC ELSD Area: | 8060927 | | |
| Corrected for | | ELSD | 97.81 | 0.72 | 1.37 | 0.10 | 7.3 | | 3.5 |
| Volatiles Loss | | 500 nm | 38.03 | 20.73 | 35.48 | 5.77 | 3.6 | 0.93 | |
| | | 700 nm | 26.80 | 22.67 | 40.73 | 9.80 | 2.3 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 1.72 | ELSD | 8.66 | 21.26 | 64.17 | 5.90 | 70.07 | 1.37 | 1.73 |
| 10μ | | 500 nm | 11.93 | 23.02 | 54.05 | 10.99 | 65.04 | | |
| 0.4020 mg | | 700 nm | 8.43 | 23.05 | 55.24 | 13.28 | 68.52 | | |
| C7 Asphaltenes | 0.01 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-11 (#1st Try Layer-2) | | | | Heavy Oil Set 3 | | | | |
| | | Centrifuged Oil from Emulsion Layer | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 51.69 | ELSD | 92.55 | 1.94 | 5.23 | 0.28 | 6.9 | | 11.6 |
| 2.0008 mg | | 500 nm | 35.54 | 22.93 | 37.58 | 3.95 | 5.8 | 1.06 | |
| | | 700 nm | 25.40 | 25.71 | 42.94 | 5.95 | 4.3 | | |
| | | Whole Oil ELSD Area: 3789074 | | | | QC ELSD Area: | 7842919 | | |
| Corrected for | | ELSD | 96.40 | 0.94 | 2.53 | 0.14 | 6.9 | | 5.6 |
| Volatiles Loss | | 500 nm | 35.54 | 22.93 | 37.58 | 3.95 | 5.8 | 1.06 | |
| | | 700 nm | 25.40 | 25.71 | 42.94 | 5.95 | 4.3 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 6.92 | ELSD | 15.61 | 21.42 | 60.71 | 2.27 | 62.98 | 4.42 | 7.02 |
| 10μ | | 500 nm | 15.73 | 28.25 | 52.08 | 3.94 | 56.02 | | |
| 0.4024 mg | | 700 nm | 10.84 | 28.98 | 55.15 | 5.03 | 60.18 | | |
| C7 Asphaltenes | 0.10 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-11 (#1st Try Layer-2) | | | | Heavy Oil Set 3 | | | | |
| | | Oil Residue from Evaporated Centrifuged Water Fraction from Emulsion Layer | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 79.21 | ELSD | 65.47 | 5.77 | 16.92 | 11.84 | 0.5 | | 51.3 |
| 0.4633 mg | | 500 nm | 32.71 | 25.12 | 35.35 | 6.82 | 3.7 | 1.08 | |
| | | 700 nm | 27.26 | 27.02 | 35.92 | 9.81 | 2.8 | | |
| | | Whole Oil 2 mg ELSD Area: 1630870 | | | | QC ELSD Area: | 7842919 | | |
| Corrected for | | ELSD | 92.82 | 1.20 | 3.52 | 2.46 | 0.5 | | |
| Volatiles Loss | | 500 nm | 32.71 | 25.12 | 35.35 | 6.82 | 3.7 | | |
| | | 700 nm | 27.26 | 27.02 | 35.92 | 9.81 | 2.8 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | | ELSD | Insufficient Material | | | | | | |
| 10μ | | 500 nm | Insufficient Material | | | | | | |
| | | 700 nm | Insufficient Material | | | | | | |
| C7 Asphaltenes | | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-12 (#1st Try Layer-3) | | | | Heavy Oil Set 3 | | | | |
| | | Whole Sample Shaken | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 69.39 | ELSD | 92.48 | 2.47 | 4.70 | 0.34 | 7.3 | | 11.7 |
| 2.0006 mg | | 500 nm | 35.89 | 21.60 | 36.82 | 5.69 | 3.8 | 1.03 | |
| | | 700 nm | 23.98 | 24.29 | 43.47 | 8.26 | 2.9 | | |
| | | Whole Oil ELSD Area: 2310376 | | | | QC ELSD Area: | 7547625 | | |
| Corrected for | | ELSD | 97.70 | 0.76 | 1.44 | 0.10 | 7.3 | | 3.6 |
| Volatiles Loss | | 500 nm | 35.89 | 21.60 | 36.82 | 5.69 | 3.8 | 1.03 | |
| | | 700 nm | 23.98 | 24.29 | 43.47 | 8.26 | 2.9 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 2.95 | ELSD | 7.64 | 22.72 | 62.75 | 6.89 | 69.64 | 2.05 | 2.98 |
| 10μ | | 500 nm | 11.24 | 22.00 | 52.78 | 13.99 | 66.77 | | |
| 0.4006 mg | | 700 nm | 6.12 | 19.94 | 50.67 | 23.27 | 73.94 | | |
| C7 Asphaltenes | 0.03 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-12 (#1st Try Layer-3) | | | | Heavy Oil Set 3 | | | | |
| | | Whole Emulsion Layer Shaken | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 69.47 | ELSD | 92.95 | 2.33 | 4.38 | 0.34 | 6.9 | | 11.5 |
| 2.0000 mg | | 500 nm | 38.49 | 20.98 | 34.78 | 5.74 | 3.7 | 0.90 | |
| | | 700 nm | 26.92 | 23.44 | 40.18 | 9.46 | 2.5 | | |
| | | Whole Oil ELSD Area: 2461011 | | | | QC ELSD Area: | 8060927 | | |
| Corrected for | | ELSD | 97.85 | 0.71 | 1.34 | 0.10 | 6.9 | | 3.5 |
| Volatiles Loss | | 500 nm | 38.49 | 20.98 | 34.78 | 5.74 | 3.7 | 0.90 | |
| | | 700 nm | 26.92 | 23.44 | 40.18 | 9.46 | 2.5 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 2.00 | ELSD | 12.05 | 21.97 | 58.69 | 7.29 | 65.98 | 1.29 | 2.02 |
| 10μ | | 500 nm | | 13.42 | 23.64 | 49.85 | 13.08 | 62.93 | |
| .4020 mg | | 700 nm | 9.28 | 23.68 | 50.75 | 16.28 | 67.03 | | |
| C7 Asphaltenes | 0.02 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-12 (#1st Try Layer-3) | | | | Heavy Oil Set 3 | | | | |
| | | Emulsoin Centrifuged Oil Fraction | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 69.11 | ELSD | 91.75 | 2.34 | 5.44 | 0.47 | 5.0 | | 12.6 |
| 2.0012 mg | | 500 nm | 34.64 | 21.79 | 37.78 | 5.79 | 3.8 | 1.09 | |
| | | 700 nm | 24.22 | 24.30 | 43.18 | 8.29 | 2.9 | | |
| | | Whole Oil ELSD Area: 2602764 | | | | QC ELSD Area: | 8427234 | | |
| Corrected for | | ELSD | 97.45 | 0.72 | 1.68 | 0.15 | 5.0 | | |
| Volatiles Loss | | 500 nm | 34.64 | 21.79 | 37.78 | 5.79 | 3.8 | | |
| | | 700 nm | 24.22 | 24.30 | 43.18 | 8.29 | 2.9 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 5.95 | ELSD | 12.95 | 27.08 | 57.51 | 2.46 | 59.97 | 3.57 | 5.97 |
| 10μ | | 500 nm | 13.74 | 30.73 | 51.39 | 4.13 | 55.52 | | |
| 2.0036 mg | | 700 nm | 8.86 | 31.43 | 54.44 | 5.27 | 59.71 | | |
| C7 Asphaltenes | 0.024 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-12 (#1st Try Layer-3) | | | | Heavy Oil Set 3 | | | | |
| | | Oil Residue from Evaporated Centrifuged Water Fraction from Emulsion Layer | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 56.33 | ELSD | 85.65 | 3.77 | 7.40 | 3.18 | 1.2 | | 22.9 |
| 1.1087 mg | | 500 nm | 37.36 | 25.86 | 32.31 | 4.48 | 5.8 | 0.86 | |
| | | 700 nm | 27.04 | 28.81 | 37.41 | 6.74 | 4.3 | | |
| | | Whole Oil 2 mg ELSD Area: 3680435 | | | | QC ELSD Area: | 8427234 | | |
| Corrected for | | ELSD | 93.73 | 1.65 | 3.23 | 1.39 | 1.2 | | 10.0 |
| Volatiles Loss | | 500 nm | 37.36 | 25.86 | 32.31 | 4.48 | 5.8 | 0.86 | |
| | | 700 nm | 27.04 | 28.81 | 37.41 | 6.74 | 4.3 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | | ELSD | Insufficient Material | | | | | | |
| 10μ | | 500 nm | Insufficient Material | | | | | | |
| | | 700 nm | Insufficient Material | | | | | | |
| C7 Asphaltenes | | | ELSD | | | | Insufficient Material | | |
| 0.45μ | | | 500 nm | | | | Insufficient Material | | |
| | | | 700 nm | | | | Insufficient Material | | |
| Sample: | WRI 1338-131-13 (#1st Try layer-4) | | | | Heavy Oil Set 3 | | | | |
| | | Whole Sample Shaken | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 73.87 | ELSD | 91.94 | 2.73 | 4.96 | 0.36 | 7.6 | | 12.1 |
| 2.0000 mg | | 500 nm | 33.44 | 22.99 | 38.13 | 5.45 | 4.2 | 1.14 | |
| | | 700 nm | 21.55 | 25.27 | 44.67 | 8.51 | 3.0 | | |
| | | Whole Oil ELSD Area: 1972189 | | | | QC ELSD Area: | 7547625 | | |
| Corrected for | | ELSD | 97.89 | 0.71 | 1.30 | 0.09 | 7.6 | | 3.2 |
| Volatiles Loss | | 500 nm | 33.44 | 22.99 | 38.13 | 5.45 | 4.2 | 1.14 | |
| | | 700 nm | 21.55 | 25.27 | 44.67 | 8.51 | 3.0 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 3.29 | ELSD | 6.60 | 18.02 | 70.64 | 4.74 | 75.38 | 2.48 | 3.31 |
| 10μ | | 500 nm | 11.83 | 24.06 | 55.37 | 8.74 | 64.11 | | |
| 0.4028 mg | | 700 nm | 8.01 | 23.83 | 56.69 | 11.47 | 68.16 | | |
| C7 Asphaltenes | 0.02 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-13 (#1st Try Layer-4) | | | | Heavy Oil Set 3 | | | | |
| | | Whole Emulsion Layer Shaken | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 62.98 | ELSD | 92.97 | 2.26 | 4.50 | 0.27 | 8.4 | | 11.3 |
| 2.0000 mg | | 500 nm | 37.73 | 21.06 | 35.91 | 5.29 | 4.0 | 0.95 | |
| | | 700 nm | 26.28 | 23.63 | 42.09 | 8.00 | 3.0 | | |
| | | Whole Oil ELSD Area: 2983869 | | | | QC ELSD Area: | 8060927 | | |
| Corrected for | | ELSD | 97.40 | 0.84 | 1.67 | 0.10 | 8.4 | | 4.2 |
| Volatiles Loss | | 500 nm | 37.73 | 21.06 | 35.91 | 5.29 | 4.0 | 0.95 | |
| | | 700 nm | 26.28 | 23.63 | 42.09 | 8.00 | 3.0 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 4.88 | ELSD | 12.38 | 20.80 | 65.00 | 1.82 | 66.82 | 1.31 | 4.89 |
| 10μ | | 500 nm | 12.36 | 26.67 | 57.21 | 3.75 | 60.96 | | |
| .4004 mg | | 700 nm | 8.10 | 27.25 | 60.02 | 4.63 | 64.65 | | |
| C7 Asphaltenes | 0.01 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-13 (#1st Try Layer-4) | | | | Heavy Oil Set 3 | | | | |
| | | Emulsion Centrifuged Oil Fraction | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 33.04 | ELSD | 92.10 | 2.50 | 5.16 | 0.25 | 10.0 | | 12.4 |
| 2.0010 mg | | 500 nm | 36.15 | 22.64 | 37.21 | 4.00 | 5.7 | 1.03 | |
| | | 700 nm | 25.31 | 25.62 | 43.29 | 5.78 | 4.4 | | |
| | | Whole Oil ELSD Area: 5642749 | | | | QC ELSD Area: | 8427234 | | |
| Corrected for | | ELSD | 94.71 | 1.67 | 3.46 | 0.17 | 10.0 | | |
| Volatiles Loss | | 500 nm | 36.15 | 22.64 | 37.21 | 4.00 | 5.7 | | |
| | | 700 nm | 25.31 | 25.62 | 43.29 | 5.78 | 4.4 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 9.0585 | ELSD | 20.48 | 23.96 | 53.87 | 1.68 | 55.55 | 5.03 | 9.09 |
| 10μ | | 500 nm | 15.18 | 29.52 | 51.55 | 3.75 | 55.30 | | |
| 2.0004 mg | | 700 nm | 10.61 | 30.45 | 54.21 | 4.72 | 58.93 | | |
| C7 Asphaltenes | 0.0316 | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-13 (#1st Try Layer-4) | | | | Heavy Oil Set 3 | | | | |
| | | Oil Residue from Evaporated Centrifuged Water Fraction from Emulsion Layer | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 48.87 | ELSD | 82.52 | 5.71 | 8.40 | 3.38 | 1.7 | | 29.0 |
| 0.9160 mg | | 500 nm | 39.75 | 24.80 | 32.07 | 3.39 | 7.3 | 0.81 | |
| | | 700 nm | 30.48 | 27.85 | 36.46 | 5.20 | 5.4 | | |
| | | Whole Oil 2 mg ELSD Area: 4308742 | | | | QC ELSD Area: | 8427234 | | |
| Corrected for | | ELSD | 91.06 | 2.92 | 4.29 | 1.73 | 1.7 | | 14.8 |
| Volatiles Loss | | 500 nm | 39.75 | 24.80 | 32.07 | 3.39 | 7.3 | 0.81 | |
| | | 700 nm | 30.48 | 27.85 | 36.46 | 5.20 | 5.4 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | | ELSD | Insufficient Material | | | | | | |
| 10μ | | 500 nm | Insufficient Material | | | | | | |
| | | 700 nm | Insufficient Material | | | | | | |
| C7 Asphaltenes | | ELSD | Insufficient Material | | | | | | |
| 0.45μ | | 500 nm | Insufficient Material | | | | | | |
| | | 700 nm | Insufficient Material | | | | | | |
| Sample: | WRI 1338-131-14 (#1st Try Layer-5) | | | | Heavy Oil Set 3 | | | | |
| | | Whole Sample Shaken (all oil) | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 40.24 | ELSD | 93.35 | 2.42 | 4.05 | 0.18 | 13.4 | | 10.5 |
| 2.0000 mg | | 500 nm | 36.43 | 23.71 | 35.73 | 4.13 | 5.7 | 0.98 | |
| | | 700 nm | 25.02 | 26.84 | 41.94 | 6.20 | 4.3 | | |
| | | Whole Oil ELSD Area: 4510300 | | | | QC ELSD Area: | 7547625 | | |
| Corrected for | | ELSD | 96.03 | 1.45 | 2.42 | 0.11 | 13.4 | | 6.3 |
| Volatiles Loss | | 500 nm | 36.43 | 23.71 | 35.73 | 4.13 | 5.7 | 0.98 | |
| | | 700 nm | 25.02 | 26.84 | 41.94 | 6.20 | 4.3 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 8.97 | ELSD | 12.00 | 22.50 | 64.38 | 1.11 | 65.49 | 58.70 | 9.40 |
| 10μ | | 500 nm | 13.93 | 29.30 | 52.63 | 4.14 | 56.77 | | |
| 0.4036 mg | | 700 nm | 9.34 | 31.25 | 54.34 | 5.07 | 59.41 | | |
| C7 Asphaltenes | 0.43 | ELSD | 8.45 | 12.95 | 76.79 | 1.81 | 78.60 | | |
| 0.45μ | | 500 nm | 9.10 | 20.14 | 66.11 | 4.65 | 70.76 | | |
| 0.4290 mg | | 700 nm | 5.77 | 21.03 | 68.06 | 5.14 | 73.20 | | |
| Sample: | WRI 1338-131-16 (#1st Try Layer-7) | | | | Heavy Oil Set 3 | | | | |
| | | Whole Sample Shaken (all oil) | | | | | | AD Asphalt | |
| Material/ | Wt. % ELSD | Asphaltene Determinator Area Percent | | | | | Coke Index | Aging Index | Percent |
| Amt. Inj. | Volatiles | Detector | Heptane | CyC₆ | Toluene | CH₂Cl₂:MeOH | Ratio Cy/CCl | Ratio T/H | TPA |
| Whole Oil | 37.91 | ELSD | 93.25 | 2.39 | 4.19 | 0.16 | 14.9 | | 10.5 |
| 2.0016 mg | | 500 nm | 35.85 | 23.66 | 36.55 | 3.93 | 6.0 | 1.02 | |
| | | 700 nm | 23.79 | 27.08 | 43.39 | 5.74 | 4.7 | | |
| | | Whole Oil ELSD Area: 4686428 | | | | QC ELSD Area: | 7547625 | | |
| Corrected for | | ELSD | 95.81 | 1.48 | 2.60 | 0.10 | 14.9 | | 6.5 |
| Volatiles Loss | | 500 nm | 35.85 | 23.66 | 36.55 | 3.93 | 6.0 | 1.02 | |
| | | 700 nm | 23.79 | 27.08 | 43.39 | 5.74 | 4.7 | | |
| | | Gravimetric Asphaltenes Analysis | | | | | Area Percent | Wt. % | Total |
| Material/ | Wt. % | Asphaltene Determinator Area Percent | | | | | Toluene + | Toluene + | Wt. % |
| Amt. Inj. | Whole Oil | Detector | Heptane | CyC₆ | Toluene | CH₂Cl₂:MeOH | CH₂Cl₂:MeOH | CH₂Cl₂:MeOH | Asphaltenes |
| C7 Asphaltenes | 8.98 | ELSD | 13.78 | 24.99 | 60.32 | 0.92 | 61.24 | 55.00 | 9.21 |
| 10μ | | 500 nm | 14.51 | 31.34 | 50.33 | 3.82 | 54.15 | | |
| 0.4032 mg | | 700 nm | 9.97 | 33.51 | 51.95 | 4.57 | 56.52 | | |
| C7 Asphaltenes | 0.23 | ELSD | 5.58 | 11.53 | 80.73 | 2.17 | 82.90 | | |
| 0.45μ | | 500 nm | 8.12 | 18.20 | 68.11 | 5.58 | 73.69 | | |
| 0.4550 mg | | 700 nm | 4.86 | 19.25 | 69.82 | 6.06 | 75.88 | | |

It is of further note that an oil and water emulsion is typically formed or made by vigorous agitation such as shaking, ultrasonic vibration, nozzle flow mixing, or high shear mixing of oil and water, for example. The emulsion itself typically appears as a single phase. Depending on the relative densities of the oil and water, if the emulsion is not complete, there can be oil and water also present, on top or below the emulsion phase. In certain cases, the oil is an oil that contains very condensed aromatic molecules. A solid material called asphaltenes can be precipitated from the oil using an excess of aliphatic hydrocarbon solvent, for example (or, as but one additional example, an alkane mobile phase). This solubility class of associated species consists of various chemical types, including the most aromatic and polar components of the oil. It is these most aromatic components that are believed to be the “core” or nucleating agents for attraction for other “peptizing” molecules in the oil in order to render the whole mixture stable (lowest free energy possible by most favorable arrangement of molecules). These solid asphaltenes contain an asphaltene subfraction that comprises the most aromatic molecules in the original oil. It also contains a large amount of less aromatic species that are associated with the most aromatic material. The most aromatic material is what may be referred to as the “peak 4” material (because in a successive dissolution protocol involving three dissolving solvents, where (a first solvent is initially used before dissolution to precipitate asphaltenes, the most aromatic material may (in certain embodiments) precipitate with the third dissolving (or the fourth total) solvent).

At least one embodiment of the inventive technology may be described as a method for changing the stability of an emulsion that comprises an emulsion hydrocarbon, from a first stability to a second, more desired stability, the method comprising the steps of precipitating at least a first asphaltene subfraction of a hydrocarbon and a second asphaltene subfraction of the hydrocarbon within a substantially inert stationary phase to generate precipitated asphaltenes, wherein the second asphaltene subfraction is more aromatic than the first asphaltene subfraction; dissolving at least a portion of at least the first asphaltene subfraction from the precipitated asphaltenes to generate at least one dissolved asphaltene subfraction; adding at least some of one of the at least one dissolved asphaltene subfraction to the emulsion hydrocarbon (whether it be some of the first or some of the second asphaltene subfraction); and changing the stability of the emulsion comprising the emulsion hydrocarbon from the first stability to the second, more desired stability.

In this and other aspects of the inventive technology, the emulsion hydrocarbon may comprise a hydrocarbon selected from the group consisting of: crude oils, asphalts, distillation residua, processed oils, oils processed via catalytic hydrotreating, oils processed via pyrolysis, tar sands oils, shale oils, coal oils, synthetic oils, biologically derived oils, asphaltenes, modified and unmodified asphalt binders and formulations, emulsions containing oils, bitumen, atmospheric bitumen, vacuum bitumen, coal tar, heavy oil or residuum, as but a few of many possible examples. Of course, solutions of such substances comprise such substances. It is of note that certain embodiments of the inventive technology may also have the effect of reducing fouling, or extending catalyst life (where a reduction in asphaltene content or a particular subfraction such as the 2^{nd} subfracting is achieved). Both effects may be found in applications involving oil and water emulsions, but not necessarily. Also, ranking or valuing hydrocarbons such as oils relative to their ability to create emulsions of a certain stability, or achieve desired changes in stability, are also considered an aspect of the inventive technology. Further, even where an entire asphaltene is added to an emulsion hydrocarbon to effect a stability change (typically this would effect a stability increase), one or more subfraction of such asphaltenes is considered as being added to the emulsion hydrocarbon. It is of note that industrial scale, and much smaller, even laboratory scale, applications of the inventive technology, are considered within its ambit. Determination of solubility profiles as an ancillary aspect of the core inventive technology, in addition to determination of solubility parameters as may assist in, e.g., predicting emulsion stability and/or ranking oils relative to formation thereof, may also form part of the inventive technology.

Note that in this and other aspects of the inventive technology, the emulsion may be a water and oil emulsion (e.g., a water in oil emulsion, an oil in water emulsion, a mixed emulsion, or a foam (such as an asphalt foam, which is considered a type of emulsion in this disclosure)).

Further note that the step of precipitating at least a first asphaltene subfraction of a hydrocarbon and a second asphaltene subfraction of the hydrocarbon within a substantially inert stationary phase may comprise the step of precipitating at least a first asphaltene subfraction of a hydrocarbon and a second asphaltene subfraction of the hydrocarbon within a substantially inert stationary phase that is selected from the group consisting of: oligomers or polymers of polytetrafluorethylene, PTFE, polyphenylene sulfide, silicon polymer, fluorinated polymers or elastomers, and PEEK stationary phase, as but a few examples of substantially inert stationary phases. Within a stationary phase includes but is not limited to on precipitation and/or accumulation of precipitated materials in the direct vicinity of the stationary phase, on a surface(s) of the stationary phase, and between discretized portions of a total stationary phase amount (e.g., a stationary phase bed or packing material, perhaps as established in a column).

Note that the first and second asphaltene subfractions (whether precipitated, absorbed, dissolved, or isolated) need not total the total asphaltenes; indeed, they may together amount to less than the total amount of asphaltenes (even significantly less than the total amount of asphaltenes). Not all precipitated or adsorbed subfaction amounts may be dissolved. Typically, such subfractions do not overlap. In certain embodiments, one asphaltene subfraction may be defined as those asphaltenes that have a parameter value that is above (or below and including) a certain value, while the other asphaltene subfraction may (but not of necessity) have a value (of that same parameter) that is below and including (or above) such certain value. For example, the second asphaltene subfraction may be a subfraction having a H/C ratio of less than or equal to 1.3. Other ways of describing such subfractions include: the second asphaltene subfraction may be a pre-coke asphaltene subfraction; the first asphaltene subfraction may be more resinous than the second asphaltene subfraction; the second asphaltene subfraction may be more polar than the first asphaltene subfraction; the second asphaltene subfraction may be more pericondensed than the first asphaltene subfraction; the second asphaltene subfraction may be a subfraction that is poorly soluble in a solvent (including, not dissolving at all or only in de minimus amounts) having a solubility parameter that is less than (<) 17 MPa^{1/2} (or even 16 MPa^{1/2}); the second asphaltene subfraction may be a subfraction that is poorly soluble in at least one aliphatic solvent (e.g., heptane and cyclohexane); the second asphaltene subfraction may be a subfraction that is soluble only in a solvent having a solubility parameter that is >16 MPa^{1/2}. Note also that certain subfractions are defined herein in terms of what is precipitated or adsorbed (so all asphaltenes having a H/C ratio of less than or equal to 1.3 in an original oil may be greater in amount than the second subfraction, which is all of that original oil's precipitated (or adsorbed) asphaltenes having a H/C ratio of less than or equal to 1.3). Indeed, while values of a parameter (e.g., H/C ratio) may be helpful in characterizing a subfraction, parameter measurements (e.g., of a H/C ratio) are not a required step in any embodiments (although they certainly may be made if desired). Note that the inventive technology is extremely flexible. For example, a second subfraction may be the top 22%-16% most aromatic of the precipitated or absorbed asphaltenes, while the 1^{st} subfraction may be the 10%-15% lowest aromaticity subfraction.

At times, the goal of certain aspects of the inventive technology may be to change the stability (as desired) of an existing emulsion (as opposed to using a subfraction as an ingredient during emulsion generation to generate emulsion on with the desired, second stability). As such, the step of adding at least some of one of the at least one dissolved asphaltene subfraction to an emulsion hydrocarbon may comprise the step of adding at least some of one of the at least one dissolved asphaltene subfraction to the emulsion comprising the emulsion hydrocarbon, while the emulsion hydrocarbon is part of the emulsion. In such embodiments, the emulsion may have the first stability before the step of adding at least some of one of the at least one dissolved asphaltene subfraction is performed.

In certain embodiments of the inventive technology, the second, more desired stability may be of a different emulsion type (e.g., where one desires to change an emulsion from oil in water to water in oil, that is considered a change in stability). Similarly, where one desires to generate an emulsion of a certain type (e.g., oil in water), where that type would be different (e.g., water in oil) without addition of some or all of a certain subfraction, then that also is considered changing of stability.

In those embodiments involving precipitation, and where the second, more desired stability is greater than the first stability, the step of dissolving may further comprise the step of dissolving at least a portion the second asphaltene subfraction to generate a dissolved second asphaltene subfraction. Such step itself may be performed after the first asphaltene subfraction is dissolved. As explained, particularly when the goal is to change the stability (as desired) of an existing emulsion, the step of adding at least some of one of the at least one dissolved asphaltene subfraction to the emulsion may comprise the step of adding at least some of the dissolved second asphaltene subfraction to the emulsion. As should be understood, the second asphaltene subfraction has the effect of stabilizing emulsions, or causing the emulsion to shift from a first type to a second type, or perhaps even causing the emulsion to form where it would not otherwise.

In those embodiments involving precipitation, and where the second, more desired stability is less than the first stability, the step of dissolving may comprise dissolving at least a portion of the first asphaltene subfraction from the precipitated asphaltenes to generate a dissolved first asphaltene subfraction. To achieve the change in destabilization of the emulsion as desired (whether that be destabilizing the emulsion (which includes breaking the emulsion), and particularly where the goal is to change the stability of an existing (already generated) emulsion), the step of adding at least some of one of the at least one dissolved asphaltene subfraction to the emulsion may comprise the step of adding at least some of the dissolved first asphaltene subfraction to the emulsion. Note that even where the goal is to destabilize an emulsion, the method may further comprise the step of dissolving at least a portion of the second asphaltene subfraction to generate a dissolved second asphaltene subfraction; such second subfraction may be used in other applications to achieve, e.g., a stabilization of an emulsion.

As mentioned, the method may be a method for generating the emulsion to have the desired, second stability instead of the first stability (even if such first stability is a “destability”, meaning absence of emulsion formulation). One way of describing certain embodiments may be adding a subfraction amount to an emulsion hydrocarbon (any hydrocarbon that will, that may (e.g., without addition of an appropriate subfraction), or that does form part of a hydrocarbon emulsion) during or before emulsion formation (emulsion generation) so that the stability of the generated emulsion will be different from what the stability would have been without such subfraction addition. Again, note that a non-emulsion is considered a destabilized emulsion; as such, certain embodiments of the inventive technology may involve adding a first subfraction to an emulsion oil to prevent such emulsion oil from becoming part of an emulsion during, e.g., agitation with water. Often, however, a second subfraction may be added as part of the emulsion recipe's ingredients in proper amount to render an emulsion (where it otherwise would not occur), or to render an emulsion with a greater stability than it would otherwise have. As such, the emulsion hydrocarbon may be generally defined as the hydrocarbon that is part of an emulsion that exists, or is to be part of an emulsion that will exist, or is to be part of an emulsion that will exist if steps are not taken to destabilize it. Other applications include rendering an emulsion to be of a certain type (e.g., water in oil), which is considered a stability, than it would otherwise have.

In certain methods involving precipitation that are for generating the emulsion to have the desired, second stability instead of the first stability, where the second stability is less than the first stability, the step of dissolving may comprise the step of dissolving to generate a dissolved first asphaltene subfraction and the step of adding at least some of one of the at least one dissolved asphaltene subfraction to an emulsion hydrocarbon may comprise the step of adding at least some of one of the dissolved first asphaltene subfraction to an emulsion hydrocarbon before the emulsion hydrocarbon is part of the emulsion and before the emulsion is formed. After such addition, the method may further comprise the step of agitating the emulsion hydrocarbon with water to generate the emulsion. Adding at least some of this first subfraction to an emulsion hydrocarbon may lower the stability of the generated emulsion as compared to what it would be without performance of the steps of adding (and changing). Particularly in those embodiments involving precipitation and re-dissolution techniques, the step of dissolving may further comprise the step of dissolving at least a portion of the second asphaltene subfraction to generate a dissolved second asphaltene subfraction. As mentioned and as is discussed elsewhere, at least some of the dissolved second apshaltene subfraction may be added to the emulsion hydrocarbon to increase the stability of the generated emulsion above what it would be without performance of the steps of adding and changing (this includes rendering an emulsion that otherwise would not be generated). As mentioned, adding at least a portion of the dissolved first asphaltene subfraction could have the effect of making the emulsion less stable as compared to what it would otherwise be (or could have the effect of the emulsion not even forming in the first place).

In certain embodiments (particularly those involving preciptitation of asphaltenes), the step of dissolving may involve the step of dissolving at least a portion of the second asphaltene subfraction to generate a dissolved second asphaltene subfraction. This step may be performed after the at least a portion of the first asphaltene subfraction is dissolved (particularly in redissolution embodiments, or embodiments involing a successive dissolution protocol). As mentioned, adding at least a portion of such dissolved second asphaltene subfraction to the emulsion hydrocarbon (whether when that hydrocarbon is part of an emulsion or before it's part of an emulsion), may have the effect of increasing emulsion stability. A few examples of possible amounts to add may be as indicated in the figures or tables supplied herewith. In redissolution protocols, the step of dissolving at least a portion of at least the first asphaltene subfraction from the precipitated asphaltenes to generate at least one dissolved asphaltene subfraction may comprise the step of dissolving with solvents of increasing strength (e.g., with solvents (perhaps mobile phase) that increase in strength via step change, or more gradually, perhaps during continuous solvent flow (although this is not necessarily required). The increasing strength may be associated with increasing polarity; one example may be dissolving in three different stages to produce three discrete asphaltene subfractions (each associated with a peak). Such dissolution protocol may be as described in U.S. Pat. No. 7,875,464, which is incorporated herein in its entirety. Such subfractions may be of increasing aromaticity.

It is of note that certain embodiments may further comprise the step of treating (e.g., via heating) the hydrocarbon before performing the step of precipitating. This, in certain embodiments, may have the effect of improving results (perhaps resulting in sharper peaks, or more Peak 4 materials (which may be eluted with the strongest, or third solvent). As discussed in U.S. Pat. No. 7,875,464, while chromatographic equipment may be used in the precipitation and redissolution type embodiments, the method is typically not chromatographic.

It is note also that the stock of the hydrocarbon from which the asphaltenes are precipitated or adsorbed (in sorbent based technologies discussed below) may be different from, or the same as, a stock of the emulsion hydrocarbon. Regardless, certain aspects of the inventive technology, particularly those involving adding a dissolved subfraction to change the stability of an emulsion yet to be formed, may involve the step of designing the oil emulsion to have the second, more desired stability (perhaps by consulting known data relative to how much of a certain dissolved asphaltene subfraction, perhaps of a particular oil stock, effects stability, and to what degree).

As with other aspects of the inventive technology, the precipitation redissolution technique may include as part of the inventive technology the refinery or apparatus in which at least part of the method is performed; in those embodiments where emulsions are part of the hydrocarbon extraction process, the inventive technology may include a refinery that processes hydrocarbons extracted, at least in part, through use of the method. Further, the inventive technology may include the dissolved asphaltene subfraction, and the substance emulsion or non-emulsion having a lowered stability) having the second, more desired stability, in addition to the including the emulsion itself.

It is of note that emulsion stability can be measured in any manner of know ways, such as length of time for the emulsion to collapse or fall; measurements of water in an emulsion may provide information as to emulsion stability or related qualities, in certain instances.

It is of further note that the subfraction used to change the stability, whether in a precipitation and dissolution protocol, or an adsorbance protocol, need not be the most or least aromatic or most or least polar subfraction (e.g., if one wants to make a stable emulsion but wants asphaltenes that are soluble in toluene, you can use the toluene-soluble asphaltenes); sometimes the most polar and most aromatic subfraction (e.g., the Peak 4 materials (that are dissolved by the third solvent in certain embodiments, such as CH₂Cl₂) might impede emulsion formation for a particular source of oil (because sometimes in order to use this Peak 4 material, one may need to add resins to the asphaltenes to make the emulsion). Relatedly, one can eliminate the most aggregated leas soluble CH₂Cl₂-soluble fraction if it hinders emulsion stability. Note that other examples of the strongest solvent used in the successive dissolution protocol may include alkane solvent, a cycloalkane solvent, a chlorinated hydrocarbon solvent, an ether solvent, an aromatic hydrocarbon solvent, a blend of a solvent with alcohol, a blend of chlorinated hydrocarbon solvent and a C₁ to C₆ alcohol, a ketone solvent, and mixtures thereof, as just a few examples.

Particular embodiments of sorbent-based inventive technologies may be described as a method for changing the stability of an emulsion that comprises an emulsion hydrocarbon, from a first stability to a second, more desired stability, the method comprising the steps of: contacting a hydrocarbon with a sorbent (or perhaps even achieving adsorption without such contact), wherein the hydrocarbon has a first asphaltene subfraction and a second asphaltene subfraction, the second asphaltene subfraction being more aromatic than the first asphaltene subfraction; adsorbing at least one of the asphaltene subfractions onto the sorbent to generate adsorbed asphaltenes; desorbing (e.g., by dissolving) at least a portion of the adsorbed asphaltenes from the sorbent to generate an isolated asphaltene subfraction; adding the isolated asphaltene subfraction to the emulsion hydrocarbon; and changing the stability of the emulsion comprising the emulsion hydrocarbon from the first stability to the second, more desired stability (e.g., predictably, as based on data, and/or controllably, perhaps in carefully measured fashion).

The emulsion hydrocarbon, the emulsion, and the first and second asphaltene subfractions may be as described elsewhere in this disclosure. One particular aspect of the sorbent-based technology that may not be found in strict precipitation and redissolution approaches may involve selective adsorption, such as use of sorbents known to adsorb a particular subfraction (e.g., reverse phase sorbents for adsorbing a first asphaltene subfraction, and sorbents such as glass (see below for other examples) for adsorbing a second asphaltene subfraction).

As with precipitation and redissolution embodiments, one may desire to change the stability of an emulsion that is yet to be formed such that it has a stability that is different than it would be without addition of at least a portion of the isolated subfraction, or one may wish to change the stability of an existing emulsion. Regardless, the addition of at least a portion of an isolated subfraction may have effects on stability as indicated above relative to the dissolved first and second subfractions. Note that generally, the sorbent (perhaps upon consideration of what effect on stability is desired) may include a stationary phase sorbent, a solid sorbent, a fixed bed, a fluidized bed, surfaced sorbent, porous membrane sorbent, high surface energy sorbent, aromatic sorbent, highly aromatic sorbent, and sorbent that is selective to adsorption of one of the asphaltene subfractions. Sorbents that may be particularly selective to the second asphaltene subfraction include metals, steel, steel wire, steel wire coils, metal wire, metal wire coils, ceramics, zeolites, clays, silica, silica gel, limestone, glass, mesh glass, glass beads, mesh glass beads, quartz, sand, alumina, and high surface energy carbonaceous materials, as but a few examples. Generally, it may be an acid or a base (although it certainly need not be).

Note that certain sorbent-based technologies may involve the step of treating a hydrocarbon having asphaltenes therein to generate a treated hydrocarbon. Such treatment, whether involving heating and/or addition of solvent or chemical additive, may help to effect selective adsorption, or compromise selective adsorption that otherwise might occur, in known manner. For example, adding cyclohexane to oil to create a solution will render a silica sorbent less selective to the most aromatic subfraction. Solvents used to contact sorbents (thereby rinse asphaltenes of fractions thereof) (regardless of whether the sorbent is small particulate, larger particulate, bulk, gel, or has any other form) may, in particular embodiments, be as described relative to the precipitation and redissolution technologies indicated elsewhere herein. Where selective adsorption is used to adsorb only the second subfraction (or portion thereof), then a strong solvent may desorb only such subfraction; where selective adsorption is used to adsorb only the first subfraction (or portion thereof), a weaker solvent may be all that is needed to desorb the desired subfraction; where no selective adsorption is used (i.e., all or some of the entire aromaticity spectrum of the asphaltenes are adsorbed), a weaker solvent may desorb only the first subfraction (or portion thereof), while in order to isolate the second subfraction, solvents of increasing strength (e.g., polarity), perhaps in a sequential desorption or dissolution protocol, may be necessary (perhaps only one additional stronger solvent is necessary). WO2012121804, which is incorporated herein in its entirety, may present desorption methods that may be implemented in certain aspects of the inventive technology.

Note that “Schabron, J. F., A. T. Pauli, and J. F. Rovani, Jr., 2001, Molecular Weight Polarity Map for Residua Pyrolysis, Fuel, 80 (4), 529-537, incorporated herein, which relates to selective solubility of asphaltenes using different solvent, may also provide examples of certain solvents that may be used in either the precipitation-based embodiments, or the adsorption-based embodiments. Further, as mentioned elsewhere herein, selectivity can be adjusted using known techniques that involve the addition of solvents to the oil (including solution thereof) that is to be contacted with the sorbent.

Note that applications of the inventive technologies disclosed herein include but are not limited to: rag layer; emulsions formed from tar sands froth extractions; warm mix asphalt preparation, cold mix asphalt preparation, modification of asphalt viscosity properties, creating desired emulsions in enhanced oil recovery, creating water and oil emulsions for pipeline shipment, modifications of emulsion stability, emulsion-based fuel formulations; making water and oil emulsions using the second subfraction (e.g., more or very polar and pericondensed materials and/or more aromatic materials) as a emulsifying agent or co-agent; oil production, desalinization, oil refining, oil processing, asphalt emulsion formulation, enhanced oil recovery, bitumen recovery, and all applications indicated in patent application US2011/0253598 (hereby incorporated herein), as but a few examples.

Note that, relative to sorbent-based inventive technology, with certain sorbents, both the less polar and the more or most polar materials (and/or the less aromatic and the more or most aromatic materials) will adsorb, while the relatively non-polar components will either not be adsorbed, or they will be adsorbed and can be washed off/desorbed with a relatively low polarity solvent (or solvent with a low chromatographic sorbent strength). The less aromatic (and/or less polar and less pericondensed) material would be adsorbed along with the most aromatic (and/or most polar and pericondensed) materials. Then, one could desorb the less polar and pericondensed material with a solvent of less/intermediate polarity, chromatographic strength or with different chemical features than is required to desorb the most polar material. Further, it is of note that where asphaltenes (or subfraction thereof) stick to a sorbent (e.g., clay fines), then silica gel may be used to separate such asphaltenes from the clay.

Particular embodiments of an additional independent aspect of the inventive technology may be described as a method for decreasing the stability of an oil emulsion, wherein oil in the oil emulsion comprises a first asphaltene subfraction and a second asphaltene subfraction, the second asphaltene subfraction being more aromatic than the first asphaltene subfraction, the method comprising the steps of: contacting the oil emulsion with a sorbent (which may, but need not at times, be selective to adsorption of the second subfraction); adsorbing the second subfraction onto the sorbent; and decreasing the stability of the oil emulsion. The sorbent selective to adsorption of the second subfraction may comprise metals, steel, steel wire, steel wire coils, metal wire, metal wire coils, ceramics, zeolites, clays, silica, silica gel, limestone, glass, mesh glass, glass beads, mesh glass beads, quartz, sand, alumina, and high surface energy carbonaceous materials, as but a few examples. It may be high surface energy, and/or high charge. The method may further comprise the step of removing the sorbent and the second asphaltene subfraction adsorbed thereon from the oil emulsion, perhaps in order that the second subfraction be useful in an application to enhance stability of an emulsion. This step may be achieved via settling, floatation or filtration as but a few examples. In order to isolate the adsorbed second subfraction, it may be desorbed therefrom (e.g., via contact, such as by rinsing, with a solvent strong enough to desorb it).

Note that Snyder, L. R., 1968, “Principles of Adsorption Chromatography” Marcel Dekker, Inc., New York, pp. 125-131 & 155-181; and Barton, A. F., 1974, “Solubility Parameters,” Chemical Reviews, 75 (6), 731-753, each of which is hereby incorporated herein, may disclose sorbents and related data useful in sorbent selection in application of certain aspects of the inventive sorbent-based technology.

It is of note that that solubility parameters of solvents are generally additive when mixed, but when two or more solvents of different chromatographic strength are mixed, the stronger solvent has much larger effect than simply additive. Considerations when selecting solvents for solubility separations are different than selecting solvents for chromatographic separations (i.e., desorption from sorbents). So polarity might, at times, not be the only parameter which governs solvent selection.

Additionally, conventional partial precipitation-based approaches, which often simply use solvent mixtures, may provide precipitates of asphaltenes that are associated peptized complexes, whereas the inventive precipitation process may, in certain embodiments, isolate the most aromatic and/or polar and most pericondensed asphaltene subfraction species (which appear to have less associated peptized complexes and are more pure than partial precipitation materials).

The initial oil used to make the emulsion contains a certain percentage of asphaltenes as usually isolated by precipitation in a hydrocarbon solvent. In the emulsion, in certain embodiments, the most aromatic portion (peak 4) of what we call asphaltenes is enriched at the water and oil interface, and this helps stabilize the emulsion. Therefore, since these molecules are at the interface, they are no longer present in the original oil (but still may be considered a part of the emulsion or a part of the oil in the emulsion, the emulsion hydrocarbon). Other molecules that are less aromatic subfraction components of asphaltenes (if they were to be precipitated with a hydrocarbon solvent), could therefore be soluble in the oil in the emulsion and therefore would not participate in asphaltene precipitation from the emulsion oil, and this the asphaltene content of the emulsion oil will apparently decrease in greater proportion than water dilution, even when the emulsion water content is accounted for. Alternatively, one may also observe apparent increases in asphaltene content in some oils centrifuged from emulsions relative to the original oil. Indeed, the phenomenon is quite complex.

What has been observed, is that for gravimetric asphaltenes precipitated with heptane from the whole sample of oil, water, and emulsion shaken for emulsion-containing Try Layers 1, 2, 3, and 4 is that the peak 4 material in the gravimetric asphaltenes is significantly enriched relative to the material in the asphaltenes from the original or desalted oil. This is also true but to a lesser extent for the gravimetric asphaltenes from the centrifuged oils (most water removed) from the emulsions for these try layers. The presence of water seems to drive this effect. An interesting observation is that the content of the most aromatic peak 4 material in the whole emulsion oils does not seem to change significantly relative to the original oil.

Embodiments of an additional aspect of the inventive technology may be described as a method for isolating a higher aromaticity asphaltene subfraction and may comprise the steps of: forming an oil emulsion from an oil and water, the oil having a pre-emulsification concentration of the higher aromaticity asphaltene subfraction; increasing the pre-emulsification concentration to a post-emulsification concentration upon performance of the step of forming the oil emulsion, wherein the post-emulsification concentration is greater than the pre-emulsification concentration; and removing the higher aromaticity asphaltene subfraction from the emulsified oil. The step of removing may be accomplished via precipitation of asphaltenes and dissolution of at least the higher aromaticity subfraction, or via adsorption of at least the higher aromaticity subfraction and desorption of the higher aromaticity subfraction (both according to methods disclosed elsewhere in this specification as applied to the emulsion). Note that concentrations are concentrations relative to a total asphaltene content of the respective pre-emulsified or post-emulsified oil. In adsorption based sub-embodiments, the step of removing may comprise the step of contacting the oil emulsion with a sorbent onto which the higher aromaticity asphaltene subfraction adsorbs (whether selectively or not). Note that treating the emulsion to adjust selectivity (increase or reduce it) may be achieved as mentioned elsewhere in this disclosure, and according to know methods. Where adorption selective to the higher aromaticity subfraction is desired, the sorbent may be, e.g., metals, steel, steel wire, steel wire coils, metal wire, metal wire coils, ceramics, zeolites, clays, silica, silica gel, limestone, glass, mesh glass, glass beads, mesh glass beads, quartz, sand, alumina, and high surface energy carbonaceous materials. Instead of using sorbent based technology to achieve the intended subfraction removal, one may remove via precipitating asphaltenes within a substantially inert stationary phase and removing the higher aromaticity subfraction via dissolution.

As can be easily understood from the foregoing, the basic concepts of the present invention may be embodied in a variety of ways. It involves both subfraction isolation and emulsion stability changing techniques as well as devices to accomplish these functions. In this application, the monitoring techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “controller” should be understood to encompass disclosure of the act of “controlling”—whether explicitly discussed or not—and, conversely, were there effectively disclosure of the act of “controlling”, such a disclosure should be understood to encompass disclosure of a “controller” and even a “means for controlling” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function.

Any patents, publications, or other references mentioned in this application for patent are hereby incorporated by reference in their entirety. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the list of References To Be Incorporated By Reference In Accordance With The Patent Application or other information statement filed with the application are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

### U.S. PATENTS

| Patent | Kind | Pub/Issue | |
|---|---|---|---|
| Number | Code | Date | Inventor/Applicant |
| 6,773,921 | | Aug. 10, 2004 | Schabron et al. |
| 462,804 | | Dec. 9, 1986 | Maes |
| 4,493,765 | | Jan. 15, 1985 | Long, et al. |
| 5,092,983 | | Mar. 3, 1992 | Eppig, et al. |
| 5,574,215 | | Nov. 12, 1996 | Bunger, et al. |
| 5,861,228 | | Jan. 19, 1999 | Descales, et al. |
| 4,990,773 | | Feb. 5, 1991 | Supernaw et al. |
| 5,969,237 | | Oct. 19, 1999 | Jones et al. |
| 4,865,741 | | Sep. 12, 1989 | Nolte et al. |
| 4,988,446 | | Jan. 29, 1991 | Haberman et al. |
| 7,875,464 | B2 | Jan. 25, 2011 | Schabron et al. |
| 4,634,680 | | Jan. 6, 1987 | Kingsley |
| 8,241,920 | B2 | Aug. 14, 2012 | Schabron et al. |
| 8,273,581 | B2 | Sep. 25, 2012 | Schabron et al. |
| 7,875,464 | B2 | 2011 Jan. 25 | Schabron et al. |
| 4,006,077 | | 1977 Feb. 1 | Wegner et al. |
| 4,036,732 | | 1997 Jul. 19 | Iranietal. |
| 4,088,540 | | 1978 May 9 | Bunas |
| 4,101,415 | | 1978 Jul. 18 | Crowley |
| 4,125,459 | | 1978 Nov. 14 | Garwin |
| 4,395,330 | | 1983 Jul. 26 | Aubior et al. |
| 4,403,765 | | 1983 Sep. 13 | Billion et al. |
| 4,419,219 | | 1983 Dec. 6 | Long et al. |
| 4,422,926 | | 1983 Dec. 27 | Gould et al. |
| 4,424,114 | | 1984 Jan. 3 | Murrell et al. |
| 4,498,971 | | 1985 Feb. 12 | Angelov et al. |
| 4,502,944 | | 1985 Mar. 5 | Nelson |
| 4,502,950 | | 1985 Mar. 5 | Ikematsu et al. |
| 4,514,287 | | 1985 Apr. 30 | Ikematsu et al. |
| 4,514,787 | | 1985 Apr. 30 | Kaneko et al. |
| 4,525,269 | | 1985 Jun. 25 | Ikematsu et al. |
| 4,572,781 | | 1986 Feb. 25 | Krasuk et al. |
| 4,765,885 | | 1988 Aug. 23 | Sadeghi et al. |
| 4,810,367 | | 1989 Mar. 7 | Chombart et al. |
| 4,816,140 | | 1989 Mar. 28 | Trambouze et al. |
| 4,888,108 | | 1989 Dec. 19 | Farnand |
| 5,124,025 | | 1992 Jun. 23 | Kolstad et al. |
| 5,124,026 | | 1992 Jun. 23 | Taylor, et al. |
| 5,152,886 | | 1992 Oct. 06 | Paris-Marcano |
| 5,258,117 | | 1993 Nov. 2 | Kolstad et al. |
| 5,326,456 | | 1994 Jul. 5 | Brons et al. |
| 5,893,302 | | 1999 Apr. 13 | Strom |
| 5,944,010 | | 1999 Aug. 31 | Hoffschmidt et al. |
| 5,944,984 | | 1999 Aug. 31 | Benguigui et al. |
| 7,223,603 | B2 | 2007 May 29 | Rovani, Jr. et al. |
| 7,718,049 | B2 | 2007 May 18 | Strack et al. |
| 7,736,900 | B2 | 2010 Jun. 15 | Pauli, et al. |
| 7,981,277 | B2 | 2011 Jul. 19 | Subramaniau et al. |

### U.S. PATENT APPLICATION PUBLICATIONS

| Publication | Kind | Pub/Issue | |
|---|---|---|---|
| Number | Code | Date | Inventor |
| 20030211621 | A1 | Nov. 13, 2003 | Rovani et al. |
| 20110066441 | A1 | Mar. 17, 2011 | Ovalles et al. |
| 20110062058 | A1 | Mar. 17, 2011 | Rogel et al. |
| 20110120950 | A1 | May 26, 2011 | Schabron et al. |
| 20120160015 | A1 | Jun. 28, 2012 | Ovalles et al. |
| 20120016168 | A1 | Jan. 19, 2012 | Schabron et al. |
| 20030221992 | A1 | Dec. 4, 2003 | Gorbaty et al. |
| 20060113218 | A1 | Jun. 1, 2006 | Hart et al. |
| 20080230442 | A1 | Sep. 25, 2008 | Iqbal et al. |
| 20110215030 | A1 | Sep. 8, 2011 | Corscadden |
| 20110266198 | A1 | Nov. 3, 2011 | Hassan et al. |

### FOREIGN PATENT DOCUMENTS

| | Kind | | |
|---|---|---|---|
| Patent No. | Code | Country | Pub/Issue Date |
| 2011113017A2 | A2 | WO | Sep. 15, 2011 |
| 0077120A2 | A2 | WO | Dec. 21, 2000 |
| 0077120A3 | A3 | WO | Dec. 21, 2000 |
| 02063292A1 | A1 | WO | Aug. 15, 2002 |
| 03096011A1 | A1 | WO | Nov. 20, 2003 |
| 400989A | A | EP | May 12, 1990 |
| 2011032123A2 | A2 | WO | Mar. 17, 2011 |
| 2011032125A2 | A2 | WO | Mar. 17, 2011 |

### NON-PATENT LITERATURE DOCUMENTS

Nonpatent Literature Reference Document

“Energy and Environmental Profile of the US Petroleum Refining Industry,” 1988, Prepared by
Energetics Inc. for U.S. Department of Energy Office of Industrial Technologies, pp 4-5, 27, 33, 49 and 62-63.
“Standard Test Method for Molecular Weight (Relative Molecular Mass) of Hydrocarbons by
Thermoelectric Measurement of Vapor Pressure,” ASTM Designation: D 2503-82 (Reapproved 1997), 871-873.
Andersen, S. I. et al., 1991, “Aggregation of Asphaltenes as Determined by Calorimetry, ” Journal
of Colloid and Interface Science, 142, 497-502, 1991.
Barton, A. F., 1974, “Solubility Parameters,” Chemical Reviews, 75 (6), 731-753.
Bodusynski, M. M. et al., 1982, Separation of Solvent-Refined Coal into Solvent-Derived
Fractions, Analytical Chemistry, 54, 372-375.
Burrell, H., 1955, “Solubility Parameters” Interchemical Review, 3-16
Carrier, H. et al; “Acoustic method for measuring asphaltene flocculation in crude oil”, Journal
of Petroleum Science and Engineering, pp 111-117
Cartz, L., ch. 3, Utrasonic Testin, Nondestructive Testing, 1995, pp. 81-98.
Del Bianco, A. et al., 1993, Thermal Cracking of Petroleum Residues 1. Kinetic Analysis of the
Reaction. Fuel, 72 (1), 7-80.
Heithaus, J. J., 1962, “Measurement and Significance of Asphaltene Peptization,” Journal of the
Institute of Petroleum 48 (458), 45-53
Hildebrand, J. H. et al., 1970, “Regular and Related Solutions” Van Nostrand Reinhold, NY, pp
24-27, pp 152-153, pp 212-215
Jones et al. “Development of an ultrasonic oil stability monitor for the assessment of asphaltene
aggregation inhydrocarbon stream”, Proceed. Intern. Conf. Mitigat. Heat Exch. Foul. Econ.
Envir. Implic. Banff, AB, Canada, July, 1999, 84-94
Long, R. B. et al., 1989, “Studies in Petroleum Composition” Revue de Institute Francais du Petrole, abstract
Long, R. B., 1979, “The Concept of Asphaltenes” Preprints, Div. Petroleum Chemistry,
American Chemical Society, 24, 891-901
Magaril, R. Z. et al., 1968, Study of the Mechanism of Coke Formation in the Cracking of
Petroleum Resins, International Chemical Engineering 8 (4), 727.
McClements, D. J. “Ultrasonic Measurements in Particle Size Analysi”, University of
Massachusetts, Amherst, USA, Encyclopedia of Analytical Chemistry (Applications, Theory and
Instrumentation) pp 5581-5587
Pal R. et al., 1989, Viscosity/Concentration Relationships for Emulsions. Journal of Rheology, 33 (7), 1021-1045.
Pauli, A. T. 1996, “Asphalt Compatibility Testing Using the Automated Heithaus Titration Test”
Preprints, Division of Fuel Chemistry, American Chemical Society, 41 (4), 1276-1281.
Pauli, A. T. et al., 1998, Relationships Between Asphaltenes, Heithaus Compatibility Parameters,
and Asphalt Viscosity. Petrol. Science and Technology, 16 (9&10), 1125-1147.
Scatchard, G. 1931, “Equilibria in Non-Electrolyte Solutions in Relation to the Vapor Pressure
and Densities of the Components” Chemical Reviews, 321-333
Pauli, A. T. et al., “Stability and Compatibility Testing of Petroleum and Asphalt” American
Laboratory, September 2003, 2 pages
Phillips, C. R, et al. 1985, Kinetic Models for the Thermal Cracking of Athabaska Bitumen, Fuel 64(5), 678-691
Schabron, J. F. et al. “Coking indexes using the Heithaus titration and asphaltene solubility, ”
Preprints - American Chemical Society, Division of Petroleum Chemistry (1999), 44(2), 187-189
Schabron, J. F. et al., 1998, “The Solubility and Three-Dimensional Structure of Asphaltenes”
Petroleum Science and Technology, 16 (3-4), 361-376
Schabron, J. F. et al., 1999 “Petroleum Residua Solubility Parameter/Polarity Map: Stability
Studies of Residua Pyrolysis” Department of Energy Report under contract # DE-FC26-98FT40322
Task, 1.2, 24 pages.
Schabron, J. F. et al., 2000 “Deposition from Heavy Oils” Department of Energy Report under
contract # DEFC26-98FT40322, 35 pages
Schabron, J. F. et al., 2001b, Molecular Weight/Polarity Map for Residua Pyrolysis, Fuel, 80 (4), 529-537.
Schabron, J. F. et al., 2001c, Non-Pyrolytic Heat Induced Deposition from Heavy Oils, Fuel, 80 (7) 919-928.
Schabron, J. F., et al., 2002b, Residua Coke Formation Predictability Maps, Fuel, 81 (17) 2227-2240.
Schabron, J. F. et al., 2001a, Predicting Coke Formation Tendencies, Fuel, 80 (10) 1435-1446.
Schabron, J. F. et al., 2002a, Characterization of Residua During Pyrolysis, Preprints, Div. of Petroleum
Chemistry, American Chemical Society, 47 (1), 17-21.
Schabron, J. F. et al., 1993, “The Characterization of Petroleum Residua” U.S. Dept of Energy Report
under contract # DE-FC21-86MC110761, 68 pages.
Schabron, J. F. et al., 2002, “Coke Formation Process Model for Petroleum Refining Efficiency Improvement”,
US Department of Energy Report DE/FG36/01G011018, 18 pages
Schabron, J. F. et al, 2002, “Coke Formation Process Model For Petroleum Refining Efficiency Improvement”,
US Department of Energy Report under contract # DE/FG36/01G011018, 40 pages
Schabron, J. F. et al., 2004, Refinery Efficiency Improvement Ultrasonic Spectroscopy and WRI Coking Indexes,
WRI Report 04-R009 to DOE under Cooperative Agreement DE-FC26-98FT40322
Singh, I. D., V. Kothiyal, V. Ramaswamy, and R. Krishna, 1990, Characteristic Changes of Asphaltenes During
Visbreaking of North Gujarat Short Residue. Fuel, 69 (3), 28-292.
Small, P. A., 1953, “Some Factors Affecting the Solubility of Polymers” Journal of Applied Chemistry, 71-80
Snyder, L. R., 1968, “Principles of Adsorption Chromatography” Marcel Dekker, Inc., New York, pp. 125-131 and 155-181
Snyder, L. R., 1968, “Principles of Adsorption Chromatography” Marcel Dekker, Inc., New York, pp. 155-199
Snyder, L. R., 1968, “Principles of Adsorption Chromatography” Marcel Dekker, Inc., New York, 206-210
Wiehe, I. A., 1993, A Phase-Separation Kinetic Model for Coke Formation, Ind. Eng. Chem. Res., 32 (11), 244-2454.
Wiehe, I. A., 1996, “Two-Dimensional Solubility Parameter Mapping of Heavy Oils” Fuel Science and
Technology International, 14 (1&2), 289-312
Bodusynski, M. S. et al., 1987, “Composition of heavy petroleums: 1. molecular weight, hydrogen deficiency,
and heteroatom concentration as a function of atmospheric equivalent boiling point up to 1400 degrees F.”
Energy & Fuels, 1, 2-11.
Schabron, J. F., et al., 2006, “Initial studies using ultrasonic spectroscopy for monitoring changes
in residua with pyrolysis,” Fuel 85, 2093-2105.
Chiantore, Oscar and Simonelli, Alessandra, “Precipitation-redissolution Liquid
Chromatography of Styrene-ethyl Acrylate Copolymers,” Polymer Engineering and Science, August 1999,
Vol. 39 No. 8, p. 1383-1388.
Cortell, Jessica M. et al., “Infulence of Vine Vigor on Grape (Vitis vinifera L. Cv. Pino Noir) Anthrocyanins.
2. Anthocyanins and Pigmented Polymers in Wine,” J. Agric. Food Chem. 2007, 55, p. 6585-6595.
Aske, Narve et al.; “Determination of Saturate, Aromatic, Resin, and Asphaltenic (SARA) Components in
Crude Oils by Means of Infrared and Near-Infrared Spectroscopy,” Energy & Fuels, 2001, 15, 1304-1312.
Corbett, L. W., “Composition of Asphalt Based on Generic Fractionation, Using Solvent Deasphaltening,
Elution-Adsorption Chromatography, and Densimetric Characterization,” Analytical Chemistry, p. 576
McCarthy, James E. et al.; “EPA's Regulation of Coal-Fired Power: Is a “Train Wreck”
Coming?”, Congressional Research Service, CRS Report for Congress, Aug. 8, 2011, 7-5700, R41914.
“Standard Test Method for n-Heptane Insulbles1”, Designation: D 3279-97 (Reapproved 2001)
“Standard Test Method for Separation of Asphalt into Four Fractions1,” ASTM International, Designation D4124-09.
Schabron, J. F., et al., “Asphaltene Determinator Method for Automated On-Column
Precipitation and Redissolution of Pericondensed Aromatic Asphaltene Components,” Energy Fuels 2010,
24, 5984-5996, DOI: 10.102/efl00822f.
Fan, T. et al., “Rapid and Accurate SARA Analysis of Medium Gravity Crude Oils,” Energy & Fuels 2002, 16, 1571-1575.
Schabron, J. F., et al., “On-column precipitation and re-dissolution of asphaltenes in petroleum residua,” Fuel 87 (2008) 165-176.
Grizzle, Patrick L, et al., “Automated Liquid Chromatographic Compound Class Grou-Type Separation of
Crude Oils and Bitumens Using Chemically Bonded Aminosilane,” Anal. Chem. 1986, 58, 2389-2396.
Jewell, D. M. et al., “Ion-Exchange, Coordination, and Adsorption Chromatographic Separation of Heavy-
End Petroleum Distillates,” Laramie Energy Research Center, Analytical Chemistry, vol. 44, No. 8, July 1972, p 1391.
Jiang, C et al., “TLC-FID (Iatroscan) analysis of heavy oil and tar sand samples,” Organic Geochemistry 39 (2008) 1210-1214.
Karlsen, D. A. et al., “Analysis of petroleum fractions by TLC-FID: applications to petroleum reservoir
description,” Org. Geochem. Vol. 17, No. 5, pp. 603-617, 1991.
Kharrat, A. et al., “Issues with Comparing SARA Methodologies,” Energy & Fuels 2007, 21, 3618-3621.
Masson, J-F et al., “Dynamics of Bitumen Fractions by Thin-Layer Chromatography/Flame Ionization
Detection,” Energy & Fuels 2001, 15, 955-960.
Radke, M et al., “Preparative Hydrocarbon Group Type Determination by Automated Medium Pressure
Liquid Chromatography,” Anal. Chem. 1980, 52, 406-411.
Schabron, J. F. et al.; “Petroleum Processing Efficiency Improvement,” Topical Report, May 2011
Wiehe, Irwin A. et al.; “The Oil Compatibility Model and Crude Oil Incompatibility,” Energy & Fuels 2000, 14, 56-59.
Fan, Z et al.; “Challenges in Processing Bitumens and Heavy Oils,” Prepr. Pap. -Am. Chem. Soc, Div. Petr. Chem. 2009, 54 (1), 4.
“Canada regulator approves Enbridge diluent Line,” Reuters, Business & Financial News, Feb. 19, 2008, Calgary, Alberta.
“Opportunity Crudes Report II: Technologies and Strategies for Meeting Evolving Market and Environmental Challenges,”
Hydrocarbon Publishing Company, an updated and expanded study of the 2006 report titled “Opportunity Crudes: Technical Challenges
and Economic Benefits.”
U.S. Provisional application No. 60/711,599, filed Aug. 25, 2005, entitled Rapid
Determination of Asphaltenes and the Cyclohexane Soluble Portion of Asphaltenes by
Automated On-Column Precipitation and Re-Dissolution; Specification 24 pages, Drawings 8 pages
USPTO Office Action for patent application No. 11/510,491 dated Jul. 13, 2009.
USPTO Office Action for patent application No. 11/510,491 dated Sep. 3, 2010.
USPTO Office Action for patent application No. 11/510,491 dated Mar. 30, 2011.
USPTO Notice of Allowance for patent application No. 11/510,491 dated Nov. 17, 2010
USPTO Supplemental Notice of Allowance for patent application No. 11/510,491 dated Dec. 9, 2010.
http://www.specialchem4adhesives.com/resources, Determining Critical Surface Tension of
Solid Substrates, printed Sep. 13, 2011, 3 pages
Energy Information Administration/Capacity Report 2001
Robinson, P. R., Petroleum Processing Overview, Practical Advances in Petroleum Processing 2006: 1-78
Rogel, E. et al. Asphaltene Stability in Processed Samples using Solubility Profile Analysis, Prepr. Pap.-Am.
Chem. Soc. Div. Pet. Chem. 2011, 56(1), 3
Ovalles, C. et al. Characterization and Preparative Separation of Heavy Crude Oils, their fractions and thermally
Cracked Products by the Asphaltene solubility Fractions Method, Prepr. Pap.-Am. Chem. Soc. Div. Pet. Chem. 2011, 56(1), 8
Schabron J. F. et al., Total Pericondensed Aromatic (TPA) Determination as an Alternative to Gravimetric
Asphaltenes, Prepr. Pap.-Am. Chem. Soc. Div. Pet. Chem. 20011, 56(1), 38
Rogel, E. et al. Determination of Asphaltenes in Crude Oil and Petroleum Products by the on Column
Precipitation Method, Energy Fuels 2009, 23, 4515-4521
Ovalles, C. et al. Characterization of Heavy Crude Oils, Their Fractions, and Hydrovisbroken Products by
the Asphaltene Solubility Fraction Method, dx.doi.org/10.1021/ef201499f | Energy Fuels 2012, 26,
549-556, Published: Dec. 7, 2011
Lopez-Linares, F. et al. Adsorption of Athabasca Vacuum Residues and Their Visbroken Products over
Macroporous Solids: Influence of Their Molecular Characteristics,
dx.doi.org/10.1021/ef201047z | Energy Fuels 2011, 25, 4049-4054, Published Aug. 17, 2011
Rogel, E., Asphaltene Chemical Characterization as a Function of Solubility: Effects on Stability and Aggregation,
dx. doi.org/10.1021/ef2013979 | Energy Fuels, Published Nov. 7, 2011
Schabron, J. F. et al. The Waxphaltene Determinator Method for Automated Precipitation and Re-Dissolution
of Wax and Asphaltene Components, Energy Fuels, Article ASAP, DOI: 10.1021/ef300184s, Feb. 27, 2012
Parallel application No. 12/970,535, Office action dated Mar. 2, 2011
Parallel application No. 12/970,535, Office action dated Oct. 7, 2011
Parallel application No. 12/970,535, Office action dated Jan. 12, 2012
Parallel application No. 12/970,535, Notice of Allowance dated Jun. 8, 2012
Parallel application No. 13/243,782, Office action dated Mar. 23, 2012
Parallel application No. 13/243,782, Office action dated Jun. 27, 2012
Parallel application No. 13/243,782, Notice of Allowance dated Aug. 3, 2012
Ovalles, C. et al. Predicting Reactivity of Feedstocks to Hydroprocessing by Using Asphaltene
Characterization Techniques. Prepr. Pap.-Am. Chem. Soc, Div. Energy Fuels Chem. 2012,
57(2), 763
Rogel, E. et al. Sediment Formation in Residue Hydroconversion Processes and Its Correlation to
Asphaltene Behavior. Prepr. Pap.-Am. Chem. Soc, Div. Energy Fuels Chem. 2012, 57(2), 745
Parallel application No. 13/490,316; Office action dated Aug. 3, 2012
Schabron, J. F. Use of the Asphaltene Determinator ™ Method to Monitor Vacuum Residue
Stability to Improve Refinery Distillation Efficiency, 2011
Parallel application No. 13/490,307; Office action dated Oct. 4, 2012
McLean J. B. et al. Reactivity Screening of Feedstocks for Catgalytic Coal/Oil Co-Processing,
September 1986,
http://web.anl.gov/PCS/acsfuel/preprint%20archive/Files/31_4_ANAHEIM_09-86_0169.pdf
Mariaca-DomAnguez et al. “Reactivity of Fluid Catalytic Cracking Feedstocks as a Function of Reactive
Hydrogen Content”, Petroleum Science and Technology, 2004, Vol. 22, Issue 1-2, pp. 13-29
Johnson and Moyse, “Pretreatment of resid FCC feedstocks”, July 2004,
http://www.digitalrefining.com/article/1000161
Baker, C. A. et al. A new chromatographic procedure and its application to high polymers, J. Chem. Soc, 1956, 2352-2362
Parallel application No. 13/600,039; Office action dated Nov. 19, 2012
Parallel application No. 13/490,316; Notice of Allowance dated Dec. 10, 2012
Alvarez, G., et al., Heavy Oil-Water Interfacial Properties and Emulsion Stability: Influence of
Dilution. Energy and Fuels, 23, 294-299, 2009
Collins, S. H., et al., Adsorption of Asphaltenes and Water on Reservoir Rock Minerals, Society of
Petroleum Engineers, SPE 11800, 1983
Czarnecki, J., et al., On the Stabilization Mechanism of Water-in-Oil Emulsions in Petroleum Systems.
Energy and Fuels, 19, 2074-2079, 2005
Czarnecki, J., Stabilization of Water in Crude Oil Emulsions. Part 2, 2009, Energy and Fuels, 23, 1253
Czarnecki, J., et al., Possible Role of Asphaltenes in the Stabilization of Water-in-Crude Oil Emulsions,
Energy and Fuels, 26, ASAP article Aug. 13, 2012
Goual, L., et al.. On-column Separation of Wax and Asphaltenes in Petroleum Fluids, Energy and Fuels, 22, 4019-4028, 2008
Hannisdal, A., et al., Particle-Stabilized Emulsions: Effect of Heavy Crude Oil Components Pre-
Adsorbed onto Stabilizing Solids. Colloids and Surfaces A: Physicochem. Eng. Aspects, 276, 45-58, 2006
Hemmingsen, P. V., et al., Emulsions of Heavy Crude Oils. I: Influence of Viscosity,
Temperature, and Dilution. J. of Dispersion Sci. Tech., 26, 615
Jestin, J., et al., A Small Angle neutron Scattering Study of the Adsorbed Asphaltene layer in Water-in-Hydrocarbon
Emulsions: Structural Description Related to Stability Langmuir, 23, 10471-10478, 2007
Menon, V. B., et al., A Review of the Factors Affectoing the Stability of Solids-Stabilized Emulsions,
Separation Science and Technology, 12-13, 2131-2142, 1988
Ortiz, D. P., et al., Effect of Surfactants on Interfacial Films and Stability of Water-in- Oil Emulsions
Stabilized by Asphaltenes, J. Colloidal and Interface Science, 351 (2), 542-555, 2010
Pauli, A. T., et al., Assessment of Physical Property Prediction Based on Asphalt Average Molecular
Structures, Prepr. Pap.-Am. Chem. Soc. Div. Pet. Chem., 255-259, 2005
Peng, J., et al., Novel Magnetic Demulsifier for Water Removal from Diluted Bitumen
Emulsion, Energy and Fuels, 26, 2705-2710, 2012
Rovani, J. F., et al., Enhanced Oil Recovery: Investigation of Oil, Water, and Emulsion
Chemistry, Western Research Institute Final Report WRI-09-027, Task 73 to the U.S.
Department of Energy under Cooperative Agreement DEFC26- 98FT40323 and to the
University of Wyoming Enhanced Oil Recovery Institute, 2009
Spiecker, P. M., et al., Interfacial Rheology of Petroleum Asphaltenes at the Oil-Water Interface,
Langmuir, 20, 4022-4032, 2010, 2004
Stanford, L. A., et al., Compositional Characterization of Bitumen/Water Emulsion Films by Negative-
and Positive-Ion Electrospray Ioinization and Field Desorption/Ionization Fourier Transform Ion
Cyclotron Resonsnce Mass
Spectrometry, Energy and Fuels, 21, 963-972, 2007
Sztukowski, D. M.; et al., Asphaltene Self-Association and Water-in-Hydrocarbon Emulsions.
J. of Colloid and Interface Sci., 265, 179-186, 2003
Sztukowski, D. M., et al., Oilfield Solids and Water-in Oil Emulsion Stability, J. Colloidal and
Interface Science, 285 (2), 821-833, 2005
Transportation Research Board, Asphalt Emulsion Technology, Circular E-C102, assembled by
Delmar R. Salomon, Transportation Research Board of the National Academies, Washington, D.C., 2006
Wu, X., Investigating the Stability Mechanism of Water-in-Diluted Bitumen Emulsions through Isolation
and Characterization of the Stabilizing Materials at the Interface. Energy and Fuels, 17, 179-190, 2003
Wikipedia, 2012, Pickering Emulsions, http://en.wikipedia.org/wiki/Pickering_emulsion
Gu. G., et al. Influence of water-soluble and water-insoluble natural surface active components on the
stability of water-in-toluene-diluted bitumen emulsion, Fuel 81 (2002) 1859-1869
Honse, S. O., et al. Separation and Characterization of Asphaltenic Subfractions, Quim. Nova,
Vol. 35, No. 10, 1991-1994, 2012
Mullins, O. C. et al. Advances in Asphaltene Science and the Yen-Mullins Model,
dx.doi.org/10.1021/ef300185p | Energy Fuels 2012, 26, 3986-4003
Nordgard, E. L, et al., Behavior of Asphaltene Model Compounds at W/O Interfaces, Langmuir 2010,
26(4), 2352-2360, Published on Web Oct. 23, 2009
Spiecker, P. M., et al. Aggregation and solubility behavior of asphaltenes and their subfractions, Journal of
Colloid and Interface Science 267 (2003) 178-193
Spiecker, P. M., et al. Effects of petroleum resins on asphaltene aggregation and water-in-oil emulsion
formation, Colloids and Surfaces A: Physicochem. Eng. Aspects 220 (2003) 9-27
Wang, S. Understanding Stability of Water-in-Dilutes Bitumen Emulsions by Colloidal Force Measurements,
Thesis, Department of Chemical and Materials Engineering, Edmonton, Alterba, CA
Zhang, L. Y. et al. Asphaltene Monolayers at a Toluene/Water Interface, Energy & Fuels 2005, 19, 1330-1336
Yang, X., et al. Investigation of Subfractions of Athabasca Asphaltenes and their role in
Emulsion Stability, Energy & Fuels 2004, 18, 770-777
Hawkins, D. J., et al., Diluent availability will constrain Canada's heavy oil, bitumen
development, Oil & Gas Journal, Oct. 29, 2002, Vol. 100, Issue 44, Pg. 64
Kharrat, A. M., Characterization of Canadian heavy oils using sequential extraction approach, Energy &
Fuels 2009, 23, 828-834
Maqbool, T., P. Srikiratiwong, and H. S. Fogler, 2011, Effect of Temperature on the Precipitation Kinetics
of Asphaltenes, Energy & Fuels, 25, 694-700.
Pauli, A. T. et al., Assessment of physical property prediction based on asphalt average molecular structures,
Prepr. Pap.-Am. Chem. Soc., Div. Pet. Chem. 2005, 50(2), 255-259
Perry, G. F., 2002, Diluent Availability will Constrain Canada's Heavy Oil, Bitumen
Development, Oil and Gas Journal, 100, 44, Oct. 28, 2002.
Pratt, S., NAFTA bumps into Alberta's upgrader plans; Trade deal would prohibit the province from
reducing the flow of bitumen to the United States once it starts, Edmonton Journal, Jun. 7, 2008, Page A19
Storm, D. A., et al., Development of solid properties and thermochemistry of asphalt binders in the 25-65 degree
C. temperature range, Energy & Fuels, 1996, 10, 855-864.
Storm, D. A. et al., Rheological study of Ratawi vacuum residue in the 298-673 K temperature range,
Energy & Fuels, 1995, 9, 168-176.
U.S. Provisional application No. 61/450,515 filed 8 Mar. 2011 entitled Reduction of Heavy Oil Viscosity Using Solid Sorbents.

Thus, the applicant(s) should be understood to have support to claim and make a statement of invention to at least: i) each of the subfraction isolation devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

In addition and as to computer aspects and each aspect amenable to programming or other electronic automation, the applicant(s) should be understood to have support to claim and make a statement of invention to at least: xv) processes performed with the aid of or on a computer, machine, or computing machine as described throughout the above discussion, xvi) a programmable apparatus as described throughout the above discussion, xvii) a computer readable memory encoded with data to direct a computer comprising means or elements which function as described throughout the above discussion, xviii) a computer, machine, or computing machine configured as herein disclosed and described, xix) individual or combined subroutines and programs as herein disclosed and described, xx) a carrier medium carrying computer readable code for control of a computer to carry out separately each and every individual and combined method described herein or in any claim, xxi) a computer program to perform separately each and every individual and combined method disclosed, xxii) a computer program containing all and each combination of means for performing each and every individual and combined step disclosed, xxiii) a storage medium storing each computer program disclosed, xxiv) a signal carrying a computer program disclosed, xxv) the related methods disclosed and described, xxvi) similar, equivalent, and even implicit variations of each of these systems and methods, xxvii) those alternative designs which accomplish each of the functions shown as are disclosed and described, xxviii) those alternative designs and methods which accomplish each of the functions shown as are implicit to accomplish that which is disclosed and described, xxix) each feature, component, and step shown as separate and independent inventions, and xxx) the various combinations and permutations of each of the above.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 20 or any other claim” or the like, it could be re-drafted as dependent on claim 1, claim 15, or even claim 25 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

1-65. (canceled)

66. A method for changing the stability of an emulsion that comprises an emulsion hydrocarbon, from a first stability to a second, more desired stability, said method comprising the steps of:
contacting a hydrocarbon with a sorbent, wherein said hydrocarbon has a first asphaltene subfraction and a second asphaltene subfraction, said second asphaltene subfraction being more aromatic than said first asphaltene subfraction;
adsorbing at least one of said asphaltene subfractions onto said sorbent to generate adsorbed asphaltenes;
desorbing at least a portion of said adsorbed asphaltenes from said sorbent to generate an isolated asphaltene subfraction;
adding said isolated asphaltene subfraction to said emulsion hydrocarbon; and
changing the stability of said emulsion comprising said emulsion hydrocarbon from said first stability to said second, more desired stability.

67. The method as described in claim 66 said hydrocarbon comprises a hydrocarbon selected from the group consisting of: crude oils, asphalts, distillation residua, processed oils, oils processed via catalytic hydrotreating, oils processed via pyrolysis, tar sands oils, shale oils, coal oils, synthetic oils, biologically derived oils, modified and unmodified asphalt binders and formulations, emulsions containing oils.

68. The method as described in claim 66 wherein said emulsion is a water and oil emulsion.

69-79. (canceled)

80. The method as described in claim 66 wherein said second asphaltene subfraction is a subfraction that is poorly soluble in a solvent having a solubility parameter that is <17 MPa^{1/2}.

81-88. (canceled)

89. The method as described in claim 66 wherein said step of adsorbing at least one of said asphaltene subfractions onto a sorbent to generate adsorbed asphaltenes comprises the step of selectively adsorbing.

90. The method as described in claim 66 wherein said step of adding said isolated asphaltene subfraction to said emulsion hydrocarbon comprises the step of adding said isolated asphaltene subfraction to said emulsion comprising said emulsion hydrocarbon, while said emulsion hydrocarbon is a part of said emulsion.

91. The method as described in claim 90 wherein said step of adsorbing comprises the step of adsorbing at least said first asphaltene subfraction.

92. The method as described in claim 91 wherein said step of desorbing comprises the step of desorbing to generate an isolated first asphaltene subfraction.

93-95. (canceled)

96. The method as described in claim 90 wherein said step of adsorbing comprises the step of adsorbing at least said second asphaltene subfraction.

97. The method as described in claim 96 wherein said step of desorbing comprises the step of desorbing to generate an isolated second asphaltene subfraction.

98. The method as described in claim 97 wherein said step of adding said isolated asphaltene subfraction to said emulsion comprises the step of adding said isolated second asphaltene subfraction to said emulsion.

99. The method as described in claim 98 wherein said step of changing the stability of said emulsion comprises the step of increasing the stability of said emulsion.

100-115. (canceled)

116. The method as described in claim 66 wherein said sorbent is selected from the group consisting of: a stationary phase sorbent, a solid sorbent, a fixed bed, a fluidized bed, surfaced sorbent, porous membrane sorbent, high surface energy sorbent, aromatic sorbent, highly aromatic sorbent, sorbent that is selective to adsorption of one of said asphaltene subfractions.

117. (canceled)

118. The method as described in claim 66 wherein said sorbent is a sorbent selected from the group consisting of: metals, steel, steel wire, steel wire coils, metal wire, metal wire coils, ceramics, zeolites, clays, silica, silica gel, limestone, glass, mesh glass, glass beads, mesh glass beads, quartz, sand, alumina, and high surface energy carbonaceous materials.

119. The method as described in claim 66 wherein said sorbent is a salt, acid or base.

120. The method as described in claim 66 wherein said sorbent comprises a carbon based sorbent.

121. The method as described in claim 66 wherein said hydrocarbon comprises a hydrocarbon selected from the group consisting of bitumen, shale oil, coal oil, coal tar, biological oil, heavy oil or residuum.

122-123. (canceled)

124. The method as described in claim 66 further comprising the step of treating a hydrocarbon having asphaltenes therein to generate a treated hydrocarbon.

125. The method as described in claim 124 wherein said step of treating a hydrocarbon comprises the step of heating said hydrocarbon.

126-132. (canceled)

133. The method as described in claim 66 wherein said step of desorbing said at least a portion of said adsorbed asphaltenes from said sorbent comprises the step of rinsing said sorbent with a solvent having a solubility parameter that is greater than 16 MPa^{1/2}.

134-221. (canceled)
